SEQUENCE LISTING (22 sequences)

SEQ ID NO: 1 - DNA, 29 bp, synthetic construct
  source 1..29: mol_type = other DNA
caagcagaagacggcatacgagattagcg

SEQ ID NO: 2 - DNA, 29 bp, synthetic construct
  source 1..29: mol_type = other DNA
caagcagaagacggcatacgagatcggcg

SEQ ID NO: 3 - DNA, 29 bp, synthetic construct
  source 1..29: mol_type = other DNA
caagcagaagacggcatacgagatgcgcg

SEQ ID NO: 4 - DNA, 29 bp, synthetic construct
  source 1..29: mol_type = other DNA
caagcagaagacggcatacgagattagcg

SEQ ID NO: 5 - DNA, 13 bp, synthetic construct
  source 1..13: mol_type = other DNA
tccttggtcgccg

SEQ ID NO: 6 - DNA, 13 bp, synthetic construct
  source 1..13: mol_type = other DNA
tccttggtgcgcg

SEQ ID NO: 7 - DNA, 13 bp, synthetic construct
  source 1..13: mol_type = other DNA
gttgctcgcgccg

SEQ ID NO: 8 - DNA, 13 bp, synthetic construct
  source 1..13: mol_type = other DNA
tccttggtgcgcg

SEQ ID NO: 9 - DNA, 13 bp, synthetic construct
  source 1..13: mol_type = other DNA
gttgctcgcgccg

SEQ ID NO: 10 - DNA, 13 bp, synthetic construct
  source 1..13: mol_type = other DNA
agtcaagagcgcg

SEQ ID NO: 11 - DNA, 13 bp, synthetic construct
  source 1..13: mol_type = other DNA
agtcaagacgccg

SEQ ID NO: 12 - DNA, 13 bp, synthetic construct
  source 1..13: mol_type = other DNA
agtcaagagcgcg

SEQ ID NO: 13 - DNA, 13 bp, synthetic construct
  source 1..13: mol_type = other DNA
tccttggtcgcta

SEQ ID NO: 14 - DNA, 13 bp, synthetic construct
  source 1..13: mol_type = other DNA
tccttggtcgcat

SEQ ID NO: 15 - DNA, 13 bp, synthetic construct
  source 1..13: mol_type = other DNA
gttgctcgcgcta

SEQ ID NO: 16 - DNA, 13 bp, synthetic construct
  source 1..13: mol_type = other DNA
tccttggtcgcat

SEQ ID NO: 17 - DNA, 13 bp, synthetic construct
  source 1..13: mol_type = other DNA
gttgctcgcgcta

SEQ ID NO: 18 - DNA, 13 bp, synthetic construct
  source 1..13: mol_type = other DNA
agtcaagacgcat

SEQ ID NO: 19 - DNA, 13 bp, synthetic construct
  source 1..13: mol_type = other DNA
agtcaagacgcta

SEQ ID NO: 20 - DNA, 13 bp, synthetic construct
  source 1..13: mol_type = other DNA
agtcaagacgcat

SEQ ID NO: 21 - DNA, 29 bp, synthetic construct
  source 1..29: mol_type = other DNA
aatgatacggcgaccaccgagatctacac

SEQ ID NO: 22 - DNA, 24 bp, synthetic construct
  source 1..24: mol_type = other DNA
caagcagaagacggcatacgagat

(19) United States
(12) Patent Application Publication
(10) Pub. No.: US 2026/0049302 A1
(43) Pub. Date: Feb. 19, 2026

(54) SYSTEMS AND METHODS FOR ASSAYING VARIOUS REGIONS OF A GENOME AT DIFFERENT RESOLUTIONS

(71) Applicant: SOPHiA GENETICS S.A., Rolle (CH)

(72) Inventors: Rieke Helena KEMPFER, Lausanne (CH); Tommaso COLETTA, Lausanne (CH); Bernardo Javier FOTH, Clarens (CH); Christian POZZORINI, Lausanne (CH); Adrian WILLIG, Geneva (CH); Tamara MAAS, Morges (CH); Zhenyu XU, Nyon (CH)

(73) Assignee: SOPHiA GENETICS S.A., Rolle (CH)

(21) Appl. No.: 19/298,761

(22) Filed: Aug. 13, 2025

Related U.S. Application Data
(60) Provisional application No. 63/683,187, filed on Aug. 14, 2024.
(60) Provisional application No. 63/711,660, filed on Oct. 24, 2024.

(51) Int. Cl. C12N 15/10

(52) U.S. Cl. CPC C12N 15/1065

(57) ABSTRACT

A method for analyzing various regions of a genome at different resolutions, is disclosed herein, wherein the method comprises: producing, a whole-genome sequencing (WGS) library, wherein the WGS library is created from at least one of: DNA, RNA, and TNA; enriching, the WGS library, for each of one or more regions of interest; producing, a final sequencing library, wherein a first grouping of genomic regions are represented at a higher coverage than a second grouping of genomic regions; applying, NGS sequencing, to the final sequencing library, creating genetic data; and analyzing, the genetic data, to identify genetic markers.

### CROSS REFERENCE TO RELATED APPLICATIONS

The present application claims the benefit of U.S. Patent Application No. 63/711,660 for SYSTEMS AND METHODS FOR ASSAYING VARIOUS REGIONS OF THE GENOME AT DIFFERENT RESOLUTIONS, filed Oct. 24, 2024; and U.S. Patent Application No. 63/683,187 for METHOD FOR DIFFERENTIALLY TAGGING MULTIPLE SEQUENCING LIBRARIES DERIVED FROM ONE WHOLE GENOME SEQUENCE POOL, filed Aug. 14, 2024, the entire contents of which are incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Aug. 5, 2025, is named 1200-049US_SeqList_ST26.xml and is 20,437 bytes in size. The sequence listing contained in this .XML file is part of the specification and is hereby incorporated by reference herein in its entirety.

### FIELD OF THE PRESENT DISCLOSURE

The present disclosure is directed to systems and methods for assaying various regions of a genome at different resolutions. More specifically, the present disclosure is directed to systems and methods for assaying various regions of a genome at different resolutions capable of providing detailed information for vast portions of said genome.

### BACKGROUND

Genetic data is used in many fields, from breeding and crop sciences to epidemiology. In a medical context, access to genetic data is crucial for prenatal screenings, to gain insights into the causes of genetic diseases, inherited or somatic such as cancer, to monitor the progression of somatic diseases, or to evaluate the effects of treatments. Different types of information can be gained from analyses of individuals markers, set of genes, up to entire genomes.

For instance, genome-wide data can provide appraisals of the general state of the genome, which serve as important predictors of the likely response of some cancers to specific therapies. Conversely, detailed analyses of specific genes can help predict the individual response to various drugs, for example in the context of pharmacogenomics. Various solutions have been devised to help health practitioners access genetic information for diverse applications, many of which harness the lowering costs of high-throughput sequencing (HTS; also known as next-generation sequencing; NGS). However, NGS assays are often tailored to the targeted markers and analyses, so that no fit-all solutions currently exist.

The advent of NGS technologies has significantly decreased the cost of deoxyribonucleic acid (DNA) sequencing in the past decade. NGS has broad applications in biology and dramatically changed the way of research and diagnosis methodologies. For example, ribonucleic acid (RNA) expression profiling or DNA sequencing can only be conducted with few numbers of genes with traditional methods, such as quantitative polymerase chain reaction (qPCR) or Sanger sequencing. Even with microarrays, profiling the gene expression or identifying mutations at the whole genome level can only be implemented for organisms whose genome size is relatively small. Utilizing NGS, RNA profiling or whole genome sequencing (WGS) has become a routine practice in biological research.

Thanks to the high throughput of NGS, multiplexed methods have been developed not only to sequence more regions, but also to sequence more samples in parallel. Compared to the traditional Sanger sequencing technology, NGS enables the detection of mutations for many more samples in different genes in parallel. Due to its advantages over traditional sequencing method, NGS sequencers are now replacing Sanger in routine diagnosis. In particular, via utilization of NGS sequencers, genomic variations of individuals (germline) or of cancerous tissues (somatic) can be routinely analyzed for a number of medical applications, ranging from genetic disease diagnostic to pharmacogenomics fine-tuning of medication in precision medicine practice.

Typical NGS applications include processing multiple fragmented DNA sequence reads, which are typically short (less than 300 nucleotides in length). The resulting reads can then be compared to a reference genome by means of a number of bioinformatics methods, to identify short variants such as single nucleotide variations (SNVs) corresponding to a single nucleotide substitution, as well as short insertions and deletions (indels) of nucleotides in the DNA sequence compared to its reference.

In NGS assays, the number of sequencing reads obtained per position, known as coverage, influences the detail and quality of the information that can be extracted. A higher coverage improves statistical inference and potentially allows detecting mutations at a lower frequency, which is especially relevant for somatic diseases, such as cancer.

Indeed, cancer mutations can be present at very low frequencies in samples with a mixture of normal and tumor cells. This is exacerbated in the case of liquid biopsy, where circulating tumor DNA (ctDNA) is detected from among other cell-free DNA (cfDNA), or in the case of measurable residual disease (MRD), where small remnants of the disease are to be detected. Higher coverage is also required to resolve complex mutations, for example those involving both copy number variation (CNV) and SNVs or indels among highly similar paralogs or those occurring in genomic regions prone to high error rates, such as mononucleotide or oligonucleotide repeats. Similarly, resolving haplotypes to call star alleles, which are haplotypic combinations of mutations relevant for pharmacogenomics, requires high coverage of the candidate regions.

Given a number of sequencing reads outputted by a sequencing machine, coverage can be increased by reducing the number of samples pooled together before sequencing or by limiting the number of genomic regions that are sequenced, typically by using a target enrichment method. Many assays, therefore, focus on parts of the genome, to achieve high coverage for those regions, while limiting the sequencing efforts and thereby the associated costs.

NGS assays targeting specific genes are extremely powerful to detect mutations for diseases with clearly established candidates. However, other clinical problems require obtaining information for a large array of genes. In the case of diseases with poorly known genetic determinants, scanning numerous genes is essential to identify new candidates. In a cancer context, genotyping numerous genes helps identify passenger mutations, providing more targets to monitor cancer progression, for example via the detection of measurable residual disease (MRD).

Large panels, therefore, exist to target all protein-coding genes (whole exome sequencing—WES), thousands of clinically-relevant genes (e.g., clinical exome sequencing—CES) or hundreds of genes relevant for specific diseases (e.g., comprehensive genomic profiling—CGP). Because the coverage is a function of the total sequencing output divided by the length of the targeted genome regions, very high coverage is usually not achieved with such large panels.

Methods that combine the benefits of screening numerous genomic regions with detailed insights from some specific regions are needed to provide polyvalent assays that can be easily deployed for numerous use cases. Thus, it would be desirable to provide systems and methods for assaying various regions of a genome at different resolutions. Moreover, it would be further desirable to provide systems and methods for assaying various regions of a genome at different resolutions while simultaneously screening large portions of the genome, ranging from coding regions of thousands of genes (exomes) to whole genomes or whole transcriptomes.

It would be yet further desirable to provide systems and methods able to obtain detailed information for selected genes and genomic regions, which provide both accuracy and precision required to identify complex mutations of clinical relevance and alleles relevant to predict the response to drugs and treatments.

The present disclosure thereby provides health practitioners with the ability to gain a wide understanding of the genetics of patients through the use of streamlined, polyvalent assays. As such, the present system and method aim to provide NGS assays that combine the strengths of large panels (e.g., whole genome sequencing, whole transcriptome sequencing, whole exome sequencing, clinical exome sequencing, comprehensive genomic profiling), with those of small panels, to offer health practitioners a tool to genotype numerous genes while simultaneously obtaining high-quality data, potentially based on customized probes, for genes and markers of interest.

### SUMMARY

This summary is provided to introduce a selection of concepts in a simplified form that are further described below in the Detailed Description. This Summary is not intended to identify key features or essential features, nor is it intended to limit the scope of the claims included herewith.

Aspects of the present disclosure relate to a method for tagging one or more sequencing libraries derived from one or more DNA fragments of a DNA sample, comprising: adding an adapter to a plurality of DNA fragments of the DNA sample to obtain an initial library pool; and incorporating a sequence identifier tag to the at least one adapter of one or more subsets of sequencing libraries from the initial library pool.

Aspects of the present disclosure relate to a method further comprising denaturing the DNA fragments; hybridizing the denatured DNA fragments with primers; and amplifying the DNA fragments to obtain the initial library pool.

Aspects of the present disclosure relate to a method, wherein the sequence identifier tag is incorporated using a polymerase and primers.

Aspects of the present disclosure relate to a method, wherein the sequence identifier tag is placed between an index sequence and an at least one oligonucleotide sequence of the at least one adapter.

Aspects of the present disclosure relate to a method, wherein the primers are mismatching primers.

Aspects of the present disclosure relate to a method, wherein the at least one oligonucleotide sequence is a P7 primer and the index sequence is an i7 index.

Aspects of the present disclosure relate to a method, wherein the sequence identifier tag has between 5 to 12 base pairs.

Aspects of the present disclosure relate to a method, wherein the at least one adapter added to the DNA fragment comprises an initial sequence identifier tag, and wherein for incorporating the sequence identifier tag, the method comprises modifying the initial sequence identifier tag to the sequence identifier tag.

Aspects of the present disclosure relate to a method, wherein the initial sequence identifier tag matches the sequence identifier tag on the 5′ end, and wherein the initial sequence identifier tag differs from the sequence identifier tag by at least two bases at the 3′ end.

Aspects of the present disclosure relate to a method, further comprising: amplifying the one or more subsets of sequencing libraries; pooling and sequencing the one or more subsets of sequencing libraries; and demultiplexing each of the one or more subsets of sequencing libraries based on a corresponding sequence identifier tag.

Aspects of the present disclosure relate to a method, further comprising: producing a capture library pool of at least one targeted DNA sequences from the one or more subsets of sequencing libraries using targeted sequencing techniques; and amplifying the capture library pool.

Aspects of the present disclosure relate to a method, wherein the amplifying the capture library pool uses at least one of: (i) a first polymerase lacking 3′ to 5′ exonuclease proof-reading activity, (ii) and/or a second polymerase that is a proof-reading polymerase.

Aspects of the present disclosure relate to a method, wherein for amplifying a template DNA fragment from the capture library pool, the method comprises: maintaining a temperature of the template DNA fragment below a first temperature threshold for a first time period to activate the first polymerase; and maintaining the temperature of the template DNA fragment above a second temperature threshold for a second time period to activate the second polymerase.

Aspects of the present disclosure relate to a method, wherein the input is genomic DNA.

Aspects of the present disclosure relate to a method, wherein the input is cDNA synthesized from RNA. In such embodiments, the starting material is used to first generate a whole-transcriptome library, which is equivalent to the WGS library described throughout.

Aspects of the present disclosure relate to a method, wherein the input is cell-free DNA (cfDNA) isolated from a bodily fluid, such as blood, urine, or cerebrospinal fluid.

Aspects of the present disclosure relate to a method for distinguishing one or more sequencing libraries derived from a deoxyribonucleic acid (DNA) sample with an incorporated adapter, comprising: generating one or more reads of the one or more sequencing libraries; and distinguishing the one or more sequencing libraries based on a corresponding sequence identifier tag associated with each of the one or more sequencing libraries.

Aspects of the present disclosure relate to a method, wherein the sequence identifier tags are incorporated inside the incorporated adapter.

Aspects of the present disclosure relate to a method for analyzing various regions of a genome at different resolutions, the method comprising: producing, a WGS library, wherein the WGS library is created from at least one of DNA, cfDNA, RNA, and TNA; enriching, the WGS library, for each of one or more regions of interest to produce a capture sequencing library; wherein a first grouping of genomic regions are represented at a higher coverage than a second grouping of genomic regions; sequencing, with next generation sequencing, the pooled sequencing library, creating genetic data; and analyzing, the genetic data, to identify genetic markers.

Aspects of the present disclosure relate to a method, wherein the capture sequencing library and the WGS library are pooled prior to sequence to produce a pooled sequencing library.

Aspects of the present disclosure relate to a method, wherein sequence identifiers are integrated in the adapters and wherein the sequence identifiers differ between the capture and WGS libraries.

Aspects of the present disclosure relate to a method, wherein the sequence identifier was modified in the capture library through post-capture amplification with a mismatching primer.

Aspects of the present disclosure relate to a method, wherein the sequence identifier was modified in the WGS library through PCR amplification with a mismatching primer after aliquoting part of the WGS library to produce the capture library.

Aspects of the present disclosure relate to a method, wherein the probes used to produce the capture library include a whole exome sequencing panel, a clinical exome sequencing panel, or a comprehensive genomic profiling panel.

Aspects of the present disclosure relate to a method, wherein the probes used to produce the capture library include a small, targeted panel including genes linked to a condition of interest.

Aspects of the present disclosure relate to a method for demultiplexing one or more sequencing libraries, the method comprising: ligating at least one adapter to a DNA fragment, wherein the DNA fragment is associated with a DNA, cfDNA, RNA, or TNA sample, to obtain a WGS library; incorporating a sequence identifier tag to the at least one adapter to one or more subsets of sequencing libraries, wherein the one or more subsets of sequencing libraries is derived from the WGS library; pooling the one or more subsets of sequencing libraries; sequencing the one or more subsets of sequencing libraries; and demultiplexing each of the one or more subsets of sequencing libraries based on the corresponding sequence identifier tag.

Aspects of the present disclosure relate to a method, further comprising: denaturing the DNA fragments; hybridizing the denatured DNA fragments with primers; and amplifying the DNA fragments to obtain the first sequencing library.

Aspects of the present disclosure relate to a method, wherein the sequence identifier tag is incorporated using a polymerase and primers.

Aspects of the present disclosure relate to a method, wherein the primers are mismatching primers.

Aspects of the present disclosure relate to a method, wherein the sequence identifier tag is incorporated between an index sequence and an at least one oligonucleotide sequence of the at least one adapter.

Aspects of the present disclosure relate to a method, wherein the at least one oligonucleotide sequence is a P7 primer and the index sequence is an i7 index.

Aspects of the present disclosure relate to a method, wherein the sequence identifier tag has between 5 to 12 base pairs.

Aspects of the present disclosure relate to a method, wherein the at least one adapter added to the DNA fragment comprises an initial sequence identifier tag, and wherein for incorporating the sequence identifier tag, the method comprises modifying the initial sequence identifier tag to the sequence identifier tag.

Aspects of the present disclosure relate to a method, wherein the initial sequence identifier tag matches the sequence identifier tag on the 5′ end, and wherein the initial sequence identifier tag differs from the sequence identifier tag by at least two bases at the 3′ end.

Aspects of the present disclosure relate to a method, further comprising amplifying the one or more subsets of sequencing libraries.

Aspects of the present disclosure relate to a method, further comprising: producing a capture library pool of at least one targeted DNA sequences from the one or more subsets of sequencing libraries using targeted sequencing techniques; and amplifying the capture library pool.

Aspects of the present disclosure relate to a method, wherein the amplifying of the capture library pool uses at least one of: (i) a first polymerase lacking 3′ to 5′ exonuclease proof-reading activity, (ii) and/or a second polymerase that is a proofreading polymerase.

Aspects of the present disclosure relate to a method, wherein for amplifying a template DNA fragment from the capture library pool, the method comprises: maintaining a temperature of the template DNA fragment below a first temperature threshold for a first time period to activate the first polymerase; and maintaining the temperature of the template DNA fragment above a second temperature threshold for a second time period to activate the second polymerase.

Aspects of the present disclosure relate to a method, wherein the capture sequencing library is produced with probes comprising a whole exome sequencing panel, a clinical exome sequencing panel, or a comprehensive genomic profiling panel.

Aspects of the present disclosure relate to a method, wherein the capture sequencing library is produced with probes comprising a small panel targeting genes linked to a given condition.

Aspects of the present disclosure relate to a method, wherein the probes used to produce the capture library comprise of mixture of at least two sets of probes targeting different genomic regions.

Aspects of the present disclosure relate to a method, wherein the two sets of probes are present at different relative concentrations, so that some regions are more representing in the sequencing reads.

Aspects of the present disclosure relate to a method, wherein the genomic regions targeted by the two sets of probes overlap.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 shows representations of reads from whole genome sequencing (WGS) and targeted capture sequencing obtained as part of a single NGS assay.

FIG. 2 illustrates a flowchart of an example method for tagging one or more sequencing libraries, according to embodiments of the present disclosure.

FIGS. 3A and 3B illustrate sequences representing adapters and sequencing libraries, respectively, according to embodiments of the present disclosure.

FIG. 4 illustrates representations of a first polymerase and a second polymerase copying a template deoxyribonucleic acid (DNA) fragment, according to embodiments of the present disclosure.

FIGS. 5A and 5B illustrate flow diagrams of differentially tagging sequencing libraries in whole genome sequencing (WGS) library and capture library, according to embodiments of the present disclosure.

FIG. 6 illustrates a bar graph of distribution of reads with a first and a second sequence identifier tag, according to embodiments of the present disclosure.

FIG. 7 illustrates a bar graph for the conversion rates for different versions of a workflow for modifying an attached sequence identifier tag, according to embodiments of the present disclosure.

FIGS. 8A and 8B illustrate the number of detected that have a variant allele fraction below 10% observed with different versions of a workflow for modifying an attached sequence identifier tag, according to embodiments of the present disclosure.

FIG. 9 illustrates an example schematic representation of the workflow to go from DNA extracted from a sample to sequencing reads after subjecting the sequencing library to sequencing.

FIG. 10 illustrates an embodiment of a method to design capture probes for obtaining different coverages of different genome regions.

FIG. 11 illustrates an embodiment of a schematic of an exemplary workflow.

FIG. 12 illustrates an embodiment of a workflow where different tags are incorporated in the WGS and capture libraries.

FIG. 13 illustrates an embodiment of a workflow where different tags are incorporated in the WGS and capture libraries.

FIG. 14 illustrates an embodiment of where two sets of probes (i.e., Probes A and Probes B) may be mixed at different concentrations before being used for capture.

FIG. 15 illustrates an example of coverages obtained with an embodiment of the present disclosure involving different ratios of Probes B to Probes A (Ratio 1), for genes targeted solely by Probes A (circles), genes targeted solely by Probes B (triangles), and genes targeted by both Probes A and Probes B (squares).

FIG. 16 illustrates the coverage distribution among 50 kb windows, obtained for one sample with an embodiment of the present disclosure where Probes B are at higher concentration.

FIG. 17 illustrates an embodiment of a median coverage achieved for targets corresponding to two sets of Probes A and B mixed before capture target-enrichment.

FIG. 18 illustrates an embodiment of a rejection rate for star allele calling achieved by the systems and methods for assaying various regions of a genome at different resolutions.

FIG. 19 illustrates an embodiment of an environment in which the systems and methods of the present disclosure may be practiced.

FIG. 20 illustrates an embodiment of a block diagram of an electronic device.

### DETAILED DESCRIPTION

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The present disclosure will now be described by reference to more detailed embodiments. This present disclosure, however, may be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by those skilled in the art to which the present disclosure belongs. The terminology used in the description herein is for describing particular embodiments only and is not intended to be limiting. As used in the description and the appended claims, the singular forms ‘a,’ ‘an,’ and ‘the’ are intended to include the plural forms as well, unless the context clearly indicates otherwise.

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

All documents mentioned in this application are hereby incorporated by reference in their entirety. Any process described in this application may be performed in any order and may omit any of the steps in the process. Processes may also be combined with other processes or steps of other processes.

Unless indicated to the contrary, the numerical parameters set forth in the following specification and attached claims are approximations that may vary depending upon the desired properties sought to be obtained and thus may be modified by the term about. At the very least, and not as an attempt to limit the application of the doctrine of equivalents to the scope of the claims, each numerical parameter should be construed in light of the number of significant digits and ordinary rounding approaches.

Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

### Definitions

A “DNA sample” refers to a nucleic acid sample derived from an organism, as may be extracted for instance from a solid tumor or a fluid. The organism may be a human, an animal, a plant, fungi, or a microorganism. The nucleic acids may be found in a solid sample such as a Formalin-Fixed Paraffin-Embedded (FFPE) sample. Further, nucleic acids may be found in a fresh sample or a fresh-frozen sample. Alternatively, the nucleic acids may be found in limited quantity or low concentration, in bodily fluids (e.g., blood or plasma), as circulating free DNA (cfDNA) in bodily fluids. Circulating free DNA can include DNA shed from a tumor in a cancer patient (circulating tumor DNA; ctDNA). Alternatively, the DNA sample might be complementary DNA (cDNA) obtained by reverse transcription of RNA.

A “nucleotide sequence” or a “polynucleotide sequence” refers to the sequence of nucleotides, such as cytosine (represented by the C letter in the sequence string), thymine (represented by the T letter in the sequence string), adenine (represented by the A letter in the sequence string), guanine (represented by the G letter in the sequence string) and uracil (represented by the U letter in the sequence string) in any polymer or oligomer of nucleotides. It may be DNA or RNA, or a combination thereof. It may be found permanently or temporarily in a single-stranded or a double-stranded shape. Unless otherwise indicated, nucleic acids sequences are written left to right in 5′ to 3′ orientation.

A “primer sequence” refers to a nucleotide sequence of at least 5 nucleotides in length comprising a region of complementarity to a target DNA a part or all of which is to hybridize to a DNA template to provide a point of initiation of elongation by a polymerase as part of a process aiming to replicate and thereby amplify a DNA target, such as via polymerase chain reaction (PCR).

The term “sequencing” refers to reading a sequence of nucleotides as a string.

High throughput sequencing (HTS) or next-generation-sequencing (NGS) refers to real-time sequencing of multiple sequences in parallel, typically between 50 and a few thousand base pairs in length. Exemplary NGS technologies include those from Illumina, Ion Torrent Systems, Oxford Nanopore Technologies, Complete Genomics, Pacific Biosciences, and others. Depending on the actual technology, NGS sequencing may require sample preparation with sequencing adaptors and/or primering sites to facilitate further sequencing steps, as well as amplification steps so that multiple instances of a single parent molecule are sequenced, for instance with PCR amplification prior to delivery to flow cell in the case of sequencing by synthesis. HTS and NGS of a DNA library will produce a set of sequencing read data, which can be processed by a bioinformatics computer in a bioinformatics workflow.

Throughout the specification, “a library”, “a DNA library” or “a sequence library” refer to double strand nucleotide sequences having adapters ligated to both ends thereof. Further, variations of the term, such as “capture library pool” and “whole genome sequence library” correspond to different types of libraries obtained using different processing steps.

“Sequencing depth” or “sequencing coverage” or “depth of sequencing” refers to the number of times a targeted set of genomic regions has been sequenced. The cost of sequencing applies to one run, and pooling multiple samples within the run decreases the cost. However, the sequencing depth is divided by the number of pooled samples. Targeted panel sequencing enables focusing the sequencing efforts to parts of the genome that are of interest. As the total length of targeted regions is decreased, a high sequencing depth for the targeted regions can be reached with a lower overall number of sequencing reads. The number of samples that can be multiplexed while maintaining sequencing depth is therefore higher.

“Coverage” or “sequence read coverage” or “read coverage” refers to the number of sequencing reads that have been aligned to a genomic position or to a set of genomic positions. In general, a genomic region with a higher coverage is associated with a higher reliability in downstream genomic characterization, in particular when calling variants.

“Aligning” or “alignment” or “aligner” refers to mapping and aligning base-by-base, in a bioinformatics workflow, the sequencing reads to a reference sequence, which can be an entire reference genome. As known in bioinformatics practice, in some embodiments “alignment” methods as employed herein may also comprise certain pre-processing steps to facilitate the mapping of the sequencing reads and/or to remove irrelevant data from the reads, for instance by removing non-paired reads, and/or by trimming the adapter sequence as the end of the reads, and/or other read pre-processing filtering means. Exemplary bioinformatics data representations with different coordinate systems (absolute or relative position indexing, 0-based or 1-based, etc.) include the BED format, the GTF format, the GFF format, the SAM format, the BAM format, the VCF format, the BCF format, the Wiggle format, the GenomicRanges format, the BLAST format, the GenBank/EMBL Feature Table format, and other suitable formats.

“Variant calling” refers to the process of identifying, in a bioinformatics workflow, the sequence variants in the aligned reads relative to a reference sequence. In bioinformatics data processing, a variant is uniquely identified by its position along a chromosome (chr, pos) and its difference relative to a reference genome at this position (ref, alt). Variants may include single nucleotide polymorphisms (SNPs), also known as single nucleotide variants (SNVs), insertions or deletions (indels), copy number variants (CNVs), as well as large rearrangements, substitutions, duplications, translocations, and others. Variants may further include gene expression differences or methylation differences, depending on the starting material. Variant calling aims to be robust enough to sort out the real sequence variants from artifacts introduced via amplification and sequencing noise artifacts, for example. In a bioinformatics workflow, a variant caller may apply variant calling to produce one or more variant calls listed in any suitable format, such as Variant Calling File (VCF format). However, other file formats may be utilized. It is understood that ‘variant calling data’ is the result of variant calling analysis according to any known method of variant calling. In the described methods, variant calling data may be data obtained from sequencing of a cancer patient tumor sample. As non-limiting examples, the present method may use variant calling data obtained with a next generation sequencing targeted panel, whole exome sequencing assay, whole transcriptome sequencing assay or whole genome sequencing assay.

The term “variant” or “genomic variant” refers to a difference in a genomic sequence relative to a designated reference sequence. In bioinformatics data processing, a variant is uniquely identified based on its chromosomal position (chr, pos) and the deviation from the reference genome at that position (ref, alt). Variants may encompass single nucleotide variants (SNVs), known as single nucleotide polymorphisms (SNPs) when referring to populations, insertions or deletions (indels), copy number variants (CNVs), and structural genomic modifications such as large-scale rearrangements, duplications, translocations, fusion, differences in gene expression levels, splicing alterations, differences in methylation, etc.

The term “mutation” or “mutated gene” refers to a gene in which at least one variant has been identified that was not present in a given reference sample or point. A “mutated gene status” may be classified as “mutated” in such instances. Otherwise, said status may be denoted as “normal.” Such a classification is commonly utilized as a biomarker in cancer diagnostics and prognostics.

A “germline variant” refers to a variant inherited from at least one parent that differs from the wild-type genomic sequence as recorded in a reference database and is present in the majority of normal cells of an individual.

A “somatic variant,” also referred to as a “somatic mutation” or “somatic alteration,” denotes a genomic alteration arising in one or more somatic cells of an individual, such as those found in a tumor. Somatic variants are restricted to a subset of the cells of the individual.

A “DNA fragment” refers to a short piece of DNA resulting from the fragmentation of high molecular weight DNA. Fragmentation may have occurred naturally in the sample organism, or may have been produced artificially from a DNA fragmenting method applied to a DNA sample, for instance by mechanical shearing, sonification, enzymatic fragmentation and other methods. After fragmentation, the DNA pieces may be end repaired to ensure that each molecule possesses blunt ends. To improve ligation efficiency, an adenine may be added to each of the 3′ blunt ends of the fragmented DNA, enabling DNA fragments to be ligated to adaptors with complementary dT-overhangs.

Throughout the specification, “an adapter” refers to a short double-stranded or partially double-stranded DNA molecule. The double stranded DNA molecule may be around 10 to 100 nucleotides (base pairs), which has been designed to be ligated to a DNA fragment. The adapter may have blunt ends, sticky ends as a 3′ or a 5′ overhang, or a combination thereof. The adapter may be realized in the form of a set of oligonucleotide sequences. An adapter can be extended during the workflow, for example through the use for PCR of primers that match the adapters on their 3′ end, but are longer on their 5′ end. The adaptor may have a phosphorothioate bond before the terminal thymidine on the 3′ end to prevent an exonuclease from trimming the thymidine, thus creating a blunt end when the end of the adaptor being ligated is double-stranded.

A “partially double stranded adaptor” refers to an adaptor including both a double-stranded region and a single stranded region. The double stranded region of the adaptor contains the ligation domain, whereas the single stranded region contains the priming sequences used for subsequent library amplification, barcoding and/or sequencing. The single stranded region can either be composed of two single stranded arms, a 5′ arm and a 3′ arm, as is the case for so-called Y-shape adaptors, or the single stranded region of partially double stranded adaptor can form a hairpin or a loop, as it is the case for the so-called U-shape adaptors. The term partially double stranded adaptor refers thus both to Y-shape and U-shape adaptors or a combination thereof.

The term “A-tailing” refers to an enzymatic method for adding an adenosine nucleotide to the 3′ end of a DNA molecule.

Throughout the specification, “a sequence identifier tag,” “tags,” or “barcodes” refer to a molecular arrangement, such as a nucleic acid sequence that is fully and uniquely specified by its string of nucleotides. The tags may be used to identify DNA sequences. Likewise, a “molecular tag” or “molecular barcode” or “molecular code” or “molecular identifier” refers to a molecular arrangement such as a nucleic acid sequence which is fully and uniquely specified by its string of nucleotides.

Throughout the specification, “ligation” refers to a process of joining of separate DNA molecules. The DNA molecules may be double-stranded. For example, adapters may be joined to DNA fragments using ligation. The initial DNA molecule may be blunt-ended or may have compatible overhangs to facilitate their ligation. Ligation may be produced by various methods known to those skilled in the art, such as including, but not limited to, a ligase enzyme, performing chemical ligation, and the like.

Throughout the specification, “amplification” refers to a polynucleotide amplification reaction to produce multiple polynucleotide sequences replicated from one or more parent/template sequences. Amplification may be produced by various methods known to those skilled in the art, including, but not limited to, a polymerase chain reaction (PCR), a linear polymerase chain reaction, a nucleic acid sequence-based amplification, rolling circle amplification, and the like.

As used herein, the term “target enrichment” or “target enhancement” or “targeted enrichment” refers to a pre-sequencing step where specific regions of interest within a genome are selectively amplified or captured, increasing their concentration relative to the rest of the genome.

The term “hybridization capture” or “capture target enrichment” refers to a targeted next generation sequencing method that uses long, biotinylated oligonucleotide baits (probes) to hybridize to the regions of interest. The DNA-bait complexes are then isolated from the rest of the DNA, leading to the overrepresentation of the DNA fragments matching the baits.

The term “ligation” refers to the joining of separate double stranded DNA sequences. The latter DNA molecules may be blunt ended or may have compatible overhangs to facilitate their ligation. Ligation may be produced by various methods, for instance using a ligase enzyme, performing chemical ligation, and other methods.

“Read trimming” or “read pre-processing” refers, in a bioinformatics workflow, to the filtering out, in the sequencing reads, of a set of nucleotides at the start of the read sequence string, such as for instance the nucleotides corresponding to the adaptor sequences, to extract the real DNA fragment sequence to be analyzed.

The term “cDNA” is also known as “complementary DNA” or “copy DNA” and refers to synthetic DNA that was reverse transcribed from RNA (e.g., messenger RNA, microRNA, etc.) through a reaction using the enzyme reverse transcriptase.

The term “A-tailing” refers to an enzymatic method for adding an adenosine nucleotide to the 3′ end of a DNA molecule.

As used herein the term “DNA polymerase” refers to any enzyme that catalyzes the production or synthesis of a new DNA. DNA polymerase uses an existing DNA or RNA as a template for DNA synthesis and catalyzes the polymerization of deoxyribonucleotides alongside the template strand, which it reads. The newly synthesized DNA strand is complementary to the template strand. DNA polymerase can add free nucleotides only to the 3′-hydroxyl end of the newly forming strand. It synthesizes oligonucleotides via transfer of a nucleoside monophosphate from a nucleoside triphosphate (NTP) or deoxyribonucleoside triphosphate (dNTP) to the 3′ hydroxyl group of a growing oligonucleotide chain. This results in elongation of the new strand in a direction. DNA polymerase can only add a nucleotide onto a pre-existing 3′-OH group. So, to begin a DNA synthesis reaction, a DNA polymerase needs a primer at which it can add the first nucleotide. Suitable primers comprise RNA and DNA.

As used herein the term “proofreading DNA polymerase” refers to any DNA polymerase that is capable of correcting its errors while performing DNA synthesis. A proofreading DNA polymerase possesses a 3′ to 5′ exonuclease activity apart from its polymerase activity, and this exonuclease activity is referred here as proofreading activity. Proofreading activity of such polymerases corrects mistakes in the newly synthesized DNA. During DNA synthesis, when an incorrect base pair is recognized, the proofreading DNA polymerase reverses its direction by one base pair of DNA. The 3′ to 5′ exonuclease activity (proofreading activity) of the enzyme allows the incorrect base pair to be excised. Following base excision, the polymerase re-inserts the correct base and DNA synthesis continues.

Proofreading polymerases can be a mesophilic DNA polymerase, a thermophilic DNA polymerase, or a combination thereof. Non-limiting examples of suitable mesophilic proofreading DNA polymerases include Klenow DNA polymerase (i.e., Klenow fragment of E. coli DNA polymerase), T4 DNA polymerase, T7 DNA polymerase, phi29 DNA polymerase, or combinations thereof. Non-limiting examples of suitable thermophilic proofreading DNA polymerases include Pfu DNA polymerase, Vent DNA polymerase, Deep Vent DNA polymerase, Pwo DNA polymerase, Phusion DNA polymerase, KOD DNA polymerase, Tli DNA polymerase, Tli DNA polymerase, or combinations thereof.

As used herein, “thermophilic polymerase” or “thermophilic DNA polymerase” refers to a polymerase that has enhanced activity and/or stability at relatively high temperatures. Thermophilic nucleic acid polymerases typically have a temperature optimum of about 70-75° C. and may operate in a range of approximately 50° C. to 90° C. These enzymes are thermostable proteins. Thermostable proteins are typically stable up to a temperature of about 95° C.

A “mesophilic polymerase” refers to a polymerase functions at moderate temperatures, usually around 37° C., which is optimal for most mesophilic organisms.

As used herein the term “non-proofreading DNA polymerase” refers to any DNA polymerase that is not capable of correcting its errors while performing DNA synthesis and do not comprise exonuclease activity. Nonlimiting examples of standard, non-proofreading DNA polymerases include Taq DNA polymerase, Tth DNA polymerase, Tfl DNA polymerase, Bst DNA polymerase, or a combination thereof.

As used herein, ‘genomic instability’ refers to an increased tendency for DNA mutation characterizing some cells, such as tumor cells. Genomic instability is often the result of defects in DNA repair mechanisms, and can lead to the accumulation of a large number of extreme genomic changes, including numerical and structural modifications of large genomic regions. Genomic instability may be, for example, associated with a condition known as homologous recombination deficiency, which results from disruptions of the homologous recombination repair pathway.

As used herein, the term “TNA” refers to total nucleic acid, which encompasses all DNA and RNA molecules extracted from a given sample. TNA typically includes of mixture of DNA and RNA at various concentrations.

As used herein, the term “mismatch” or “mismatched base” refers to an instance where two non-complementary nucleotide bases are paired together within a double helix, i.e., a base incapable of forming a hydrogen bond pair with an opposite base in a target nucleic acid sequence.

As used herein, the term “multiplexing” refers to the practice of combining and sequencing multiple distinct DNA samples or targets within a single sequencing run or experiment.

As used herein, the term “demultiplexing” refers to the process of separating DNA sequences into their respective samples after they have been pooled together and sequenced as a single batch.

As used herein, the term “PCR” or “polymerase chain reaction” refers to a laboratory technique that amplifies copies of a particular DNA fragment involving a series of temperature changes known as thermal cycling. Each thermal cycle comprises three main steps: 1) denaturation, or melting, wherein the reaction mixture is heated to a high temperature to break the hydrogen bonds separating the double-stranded DNA into two single-stranded DNA templates; 2) annealing, wherein the temperature is lowered allowing the primers to bind (anneal) to their complementary sequences on the single-stranded DNA templates; and 3) extension, wherein the temperature is raised again to an optimal temperature for the DNA polymerase to extend the primers by adding deoxyribonucleotide triphosphates (dNTPs) and synthesizing new DNA strands complementary to the template.

As used herein, the term “qPCR” or “quantitative polymerase chain reaction” or “real-time PCR” or “real-time quantitative PCR” refers to a laboratory technique that combines the DNA amplification capabilities of traditional PCR with the ability to measure the amount of DNA being amplified in real time, e.g., with the use of DNA binding dyes of sequence-specific fluorescent probes.

As used herein, the term “star allele” refers to a specific, well-defined variant or haplotype of a gene, particularly those involved in drug metabolism, that has been identified and named using a standardized nomenclature system. These alleles are often associated with differences in how individuals respond to medications. Star alleles represent combinations of genetic variants which are relevant in pharmacogenomics.

As used herein, the term “low pass” or “low-pass sequencing” refers to a technique in which each base is only sequenced a few times, resulting in low coverage depth.

As used herein, the term “high pass” or “high-pass sequencing” refers to a technique in which each base is sequenced many times, resulting in high coverage depth.

As used herein, the term “large panel” or “large panel library” refers to a sequencing library comprising a broad set of genes, e.g., whole genome sequencing, whole transcriptome sequencing, whole exome sequencing, clinical exome sequencing, or comprehensive genomic profiling.

As used herein, the term “small panel” or “small panel library” refers to a sequencing library comprising a small set of genes, e.g., less than 100 genes associated with a specific condition.

### Prior Art Problems Addressed

Multiple types of sequencing libraries may be generated from subsets of DNA fragments of a DNA sample. Each type of sequencing library may be processed and sequenced differently from one another. For example, WGS gives a comprehensive view of the entire genome, while targeted sequencing focuses on specific regions of interest, allowing for deeper coverage and detailed analysis. Moreover, sequencing libraries are routinely, distinctly processed for different applications. For example, sequencing libraries identifying genome-wide patterns may require WGS libraries, while applications requiring higher coverage may require targeted enrichment of libraries.

NGS involves processing multiple DNA fragments of a DNA sample, typically by adding barcodes on both extremities of fragmented DNA, to produce a sequencing library. The sequencing library is then subjected to sequencing-by-synthesis for generating reads from one or both ends of each of a number of DNA molecules containing the added barcodes. The barcodes can then be used to sort the reads corresponding to different input samples, in a process called demultiplexing.

Multiple libraries can be labeled using sample barcodes allowing the reads originating from each library to be identified after sequencing within a single run. If different libraries contain the same sample label and are sequenced in a single sequencing run, the reads from each library cannot be distinguished from each other. Different libraries can contain the same sample barcode if they are derived from the same initial preparation after the sample barcodes have been incorporated.

Deeper insights can be gained by combining analysis obtained from differently processing libraries, such as by combining analysis from low-pass, large panels (e.g., WGS or WES) with small panels (e.g., capture-based target enrichment of small number of genes). Independently on the panel size, capture hybridization is performed on an initial whole-genome sequencing library (or whole transcriptome library when using RNA as the starting material). Different libraries can therefore be produced from the same initial whole-genome sequencing library.

However, libraries generated from the same initial whole-genome sequencing library may have to be processed on different sequencing machines, which increases cost due to the duplication of sequencing runs and, due to the need for obtaining additional numbers of DNA samples to reduce the costs by processing multiple samples per sequencing lane. Thus, there is a need for a method that allows multiple sequencing libraries generated with the same sample barcodes to be distinguished when pooled and processed using one sequencing machine.

Pooling large and small panel libraries for sequencing on a single machine presents a different set of challenges. For instance, it is not possible to identify and/or differentiate the sequencing reads originating from each preparation. Additionally, a very high relative coverage may be expected for regions targeted by probes used for preparing small panel libraries (e.g., capture libraries).

Large panel sequencing, in general, such as whole exome sequencing, clinical exome sequencing, etc., relies on a large set of capture probes designed to bind to coding regions and enrich them in the DNA preparation, called a library, that will then be subjected to sequencing. Depending on their design, capture probes can be biased for some possible alleles and thereby provide unequal coverage of the genetic makeup of the sample.

In particular, probes designed based on the reference genome will be biased for alleles matching the reference (wild-type alleles) and may therefore under detect the presence of alternative alleles, especially if these differ significantly from the reference, for example via multiple or large insertions and/or deletions. Such biases can be reduced by improving the design of capture probes, for instance by adding probes matching known alternative alleles.

Similarly, probes can be added to capture paralogs (e.g., pseudogenes) of targeted genes, for example to detect gene conversion. Probes can even be designed to specifically detect structural variants, such as the Boland inversion associated with Lynch syndrome. Additionally, probes can be added to detect important variants in non-coding fractions of the genome. Customized design of probes to improve detection of specific complex variants is not practicable for large gene panels and is therefore typically restricted to smaller targeted panels.

Some clinical applications benefit from accessing information from across the whole genome. In addition to covering all genes and their pseudogenes, whole-genome sequencing provides data on non-coding and intergenic fractions, which can increase the diagnosis yield of rare diseases, for example by identifying non-coding pathogenic mutations disrupting splicing. Besides increasing the potential candidates for diseases to non-coding fractions, genome-wide information is also useful to assess the overall state of the genome.

Genome markers, such as genomic instability (GI), are indeed actionable markers, linked to different responses to specific treatments. WGS, moreover, enables tracking dynamics of repeat element expansion, such as Alu elements that are numerous in humans. Because the total length of targeted regions is much longer in a WGS assay (about 100 times larger than in whole exome sequencing), the sequencing coverage is generally lower.

For a given investigation, health practitioners might benefit from combining customized or detailed analyses of selected genes and markers with scanning of more numerous regions at a lower coverage. Besides increasing the number of candidates for genetic diseases, such combination also increases the number of potential actionable markers and use of the data.

Some NGS assays combine WGS at low coverage with target enrichment, providing both gene-specific high-quality information and genome-wide metrics. However, these applications are limited in scope and do not provide the modularity to sequence some genic regions at moderate coverage and others at high coverage. In addition, large panels would benefit from the ability to improve and customize probe design to remove biases, capture additional alternative alleles, and detect known non-coding and structural variants. Finally, assays combining different panel sizes would benefit from the ability to tag and recognize sequencing reads originating from the different steps.

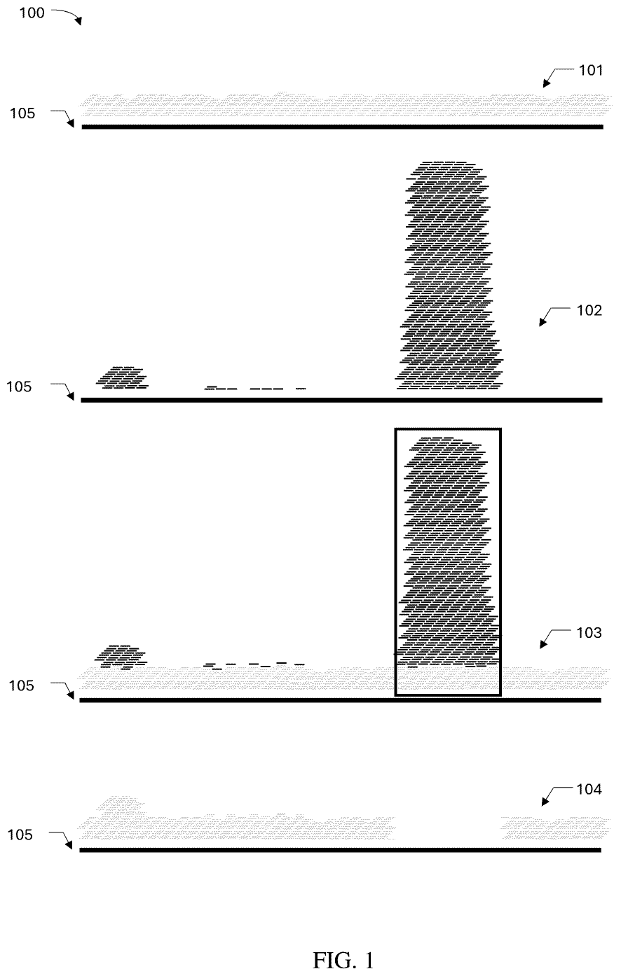

FIG. 1 illustrates a representation 100 depicting challenges presented by distribution of reads compared to a reference genome 105. The reference genome 105 is represented by a long bar at the bottom of each representation 101-104, with small bars representing aligned sequencing reads.

As shown in FIG. 1, representation 101 is indicative of reads from a large panel, e.g., WGS, which are expected to be uniformly distributed along its corresponding reference genome, with significant deviations most often indicative of copy-number variation.

Further, representation 102 is indicative of reads from a target enrichment assay, e.g., capture-based targeted sequencing. High coverage (large number of reads) is expected for the targeted gene (on the right in this example), but with possible off-target reads in other parts (on the left in this example). Off-target reads may be excluded from follow-up analyses, if they are identified as such.

Representation 103 illustrates an implementation where the reads from WGS and capture-based targeted sequencing are pooled together into a pooled sequence. As shown, if whole-genome and targeted libraries are pooled before sequencing, reads originating from each library cannot be distinguished.

Off-target reads from the targeted library can inflate the observed coverage, as visible on the left in representation 104, which may lead to the spurious inference of copy number increases. In addition, regions targeted during library preparation must be black-listed (grey box) for genome-wide coverage estimates, as shown in representation 104. Alongside capture-based targeted sequencing reads, the whole-genome sequencing reads matching the target regions are discarded from genome-wide analyses, leading to an effective data loss.

In the absence of differentially tagging the reads derived from the large and small panel libraries, all reads outside the target would be interpreted as large panel reads, while those from the small panel would all be assigned to the targeted preparation. Such challenges make pooling of multiple libraries for sequencing inefficient.

Therefore, there is a need for a method to generate multiple sequencing libraries that are distinguishable from each other. Further, there is a need to provide a method to recognize sequencing reads originating from different library types issued from the same initial large panel library after pooled sequencing. The present disclosure addresses the limitations of these conventional approaches.

The method described herein provides such an advantage by distinguishably tagging sequencing libraries prepared from the DNA fragments and by providing different sequencing depths for different parts of the genome. The present disclosure may be directed towards providing NGS assays that combine the strengths of large panels (e.g., whole genome sequencing, whole exome sequencing, clinical exome sequencing, comprehensive genomic profiling), with those of small panels (e.g., targeted enrichment sequencing), to offer health practitioners a tool to genotype numerous genes while simultaneously obtaining high-quality data, potentially based on customized probes, for genes and markers of interest.

Firstly, the disclosure may be deployed to produce a NGS assay targeting multiple genes, that can be adopted to identify and diagnose a large array of hereditary diseases, while offering increased resolution of some regions known to contain important markers that are difficult to resolve, alternative alleles that are not efficiently captured by probes based on the reference genome, important non-coding markers, or other desired features. Such an assay may be obtained by combining probes for the large panel with probes for increased resolution of specific regions.

Secondly, the disclosure may be deployed to produce a NGS assay targeting multiple genes that can be adopted to identify, diagnose, and monitor somatic diseases, such as cancer, while offering increased resolution of some regions. Such an assay may be obtained by combining probes for a large panel with probes for increased resolution of specific regions.

Probes for increased resolution of specific regions can be designed to resolve complex variants, such as those involving a combination of substitutions and insertions/deletions, those in low-diversity regions, such as mono- or di-nucleotide repeats, those involving gene fusions, and the like.

Thirdly, the disclosure may be deployed to produce a NGS assay providing a complete representation of the genome, but with increased representation of some region that are of special interest and difficult to resolve without higher coverage. The NGS assay is achieved by combining the original whole-genome sequencing library, which can be obtained using a PCR-free method, with a capture panel library, which can obtained based on a mix of different sets of probes or a single probe set. The probes set will typically target regions where identification of complex variants, such as those involving a combination of substitutions and insertions/deletions, those in low-diversity regions, such as mono- or di-nucleotide repeats, those involving gene fusions, and the like, are important or regions where allele phasing is important, for example to identify star alleles.

Additionally, the disclosure may provide the use of mismatched primers during a DNA library preparation workflow. This effectively generates a different tag in DNA molecules from an initial WGS library as compared to either a capture-based targeted library or a derived WGS library by using the mismatched primers for a secondary amplification.

To decrease the risk that proofreading DNA polymerases might correct the mismatched bases according to the template, the disclosure may provide the use of different DNA polymerases, as shown in Examples 1 and 2.

For example, a non-proofreading polymerase can be used for the entire amplification conducted post capture. Low-level noise from the lack of 3′ to 5′ exonuclease activity is then introduced into the amplified DNA molecules. The error rate of most polymerases without 3′ to 5′ exonuclease activity is about 2×10⁻⁴, which is far below the relevant limit of detection of most currently-used capture-based NGS assays, where variant fractions above 2% are required to be detected.

Additionally, a combination of two polymerases may be used. A non-proofreading polymerase may be used to copy the part of the molecules that includes the tag, ensuring the mismatch is not corrected based on the template. A second proofreading polymerase is then used to copy the rest of the DNA molecules, limiting error insertion in the sequencing reads. The activity of each polymerase is controlled temporally by changing the reaction temperature, with an increase of temperature after a given period of time disactivating the non-proofreading polymerase and activating the proofreading polymerase.

Moreover, secondary amplification can be performed by a non-proofreading polymerase. Low-pass large panel data is typically used for the detection of large copy number alterations or genomic rearrangements, which is not impacted by an elevated error rate of the polymerase. Then, the small panel library is amplified with a proofreading polymerase, as typically used in NGS assays, limiting its error rate.

Fourthly, the disclosure may be deployed to produce a NGS assay that targets genes of clinical importance for somatic diseases, such as cancer, while providing a low-coverage scan of the whole genome that can be used to infer genome-wide patterns, such as a genomic instability index. As such, large and small panel libraries can be tagged differently, so that reads originating from each can be identified from the sequencing outputs and analyzed distinctively.

In some examples, the sequencing libraries may include a first library indicative of a large panel, or WGS library and a second library indicative of a small panel library, e.g., obtained by probe-binding capture of the first library. However, it may be appreciated by those skilled in the art that the sequencing libraries may refer to any set of heterogeneously processed libraries associated with the same DNA fragments.

### Capture Probe Design

The present disclosure may be directed towards providing NGS assays that combine the strengths of large panels (e.g., whole genome sequencing, whole exome sequencing, clinical exome sequencing, comprehensive genomic profiling), with those of small panels, to offer health practitioners a tool to genotype numerous genes while simultaneously obtaining high-quality data, potentially based on customized probes, for genes and markers of interest.

In an embodiment, the systems and methods for assaying various regions of a genome at different resolutions may improve large capture panel NGS assays, ranging from whole-exome sequencing (WES) to comprehensive genomic profiling panels, by integrating the strengths of small, targeted panels.

In another embodiment, the systems and methods may enable higher coverage for some regions as part of an NGS assay covering numerous genes, up to whole genome sequencing. Indeed, the coverage typically obtained with large panels does not allow resolving variants among highly similar paralogs, variants in low-diversity regions such as mono- or di-nucleotide repeats, small copy number variants, phased haplotypes, and the like. Furthermore, the systems and methods may provide an assay capable of jointly obtaining the necessary high coverage for regions of interest and coverage of numerous other genomic regions.

In a further embodiment, the systems and methods may improve capture of some regions of a genome. For example, exome sequencing and other large panels are typically obtained by capture target enrichment, using a large number of capture probes. As these probes are generally designed based on the reference genome, they can be biased toward the wild-type allele and either capture less efficiently alternative alleles differing strongly from the reference or not capture such alternative alleles at all. These biases may be corrected by adding probes matching the alternative alleles.

Similarly, probes might be desired to capture paralogs of targeted genes (e.g. pseudogenes), capture important non-coding markers or resolve known structural variants. Finally, probes might be added to reduce problems in coverage heterogeneity. The large number of probes typical of large panels may render customization unpracticable, but the present disclosure herein may provide an efficient combination of probes for large panels with customized and/or improved probes within a single assay.

The present disclosure may be further directed to tagging of reads originating at different steps in a workflow. Some analytical tools, especially those based on coverage, may focus on reads originating from a distinct step in a data generation workflow. For instance, genome-wide coverage analyses may need to be restricted to reads originating from the WGS library, while excluding those resulting from the capture library.

In existing combined workflows, such reads are indistinguishable, but the systems and methods may provide for a method to differently tag WGS and capture reads generated as part of a single workflow. Thus, the systems and methods disclosed herein may be applied to differentially tagged reads originating from distinct capture target enrichment, pooled together before sequencing. As such, the systems and methods may provide a streamlined workflow to obtain sets of reads corresponding to different NGS assays but produced simultaneously.

The systems and methods may prepare and analyze a NGS assay so that some regions of the genome are targeted for sequencing at higher coverage than other sequenced regions. In an embodiment, the systems and methods may include the design or use of designed capture probes 1003-1004. For example, capture probes 1003-1004 may be designed to bind to genomic regions of interest. In some embodiments, a single set of probes 1003-1004 may be designed to capture regions of interest. Additionally, at least two different sets of capture probes 1003-1004 may be designed.

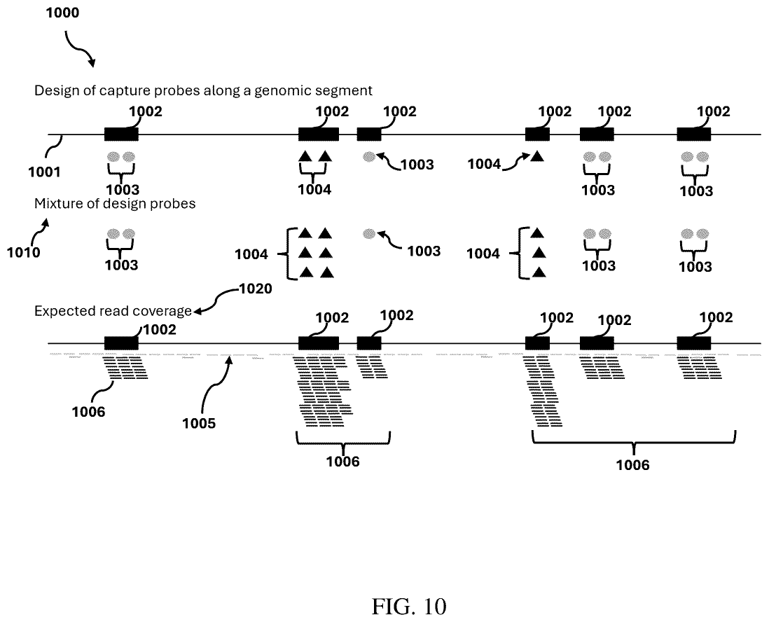

Referring to FIG. 10, capture probes 1003-1004 may be designed to target genomic regions, as shown in step 1000. In such an example, a chromosomal segment 1001 may be represented by a thin line, with genes 1002 shown as black rectangles. Putative probes 1003-1004 may be indicated with grey circles for regions targeted at a normal coverage 1003 and with black triangles for regions targeted at a higher coverage 1004. The probes may be subsequently mixed in different relative concentrations, as shown in step 1010.

For instance, the probes for regions targeted at a higher coverage 1004 (black triangles) may be three times more abundant in the final mix. The expected coverage may be a function of the relative concentration of probes 1003-1004 in the mix. Reads 1006 may be represented by short lines. Three times more reads may be expected for regions with the more abundant probes 1003-1004. In addition, some whole-genome sequencing library 1005 may be added, leading to low expected coverage 1020 across all regions, represented with grey bars.

A first set of probes 1003 may be designed to capture a large number of genomic regions 1002 and may correspond to a panel for whole-exome sequencing, clinical-exome sequencing, comprehensive genomic profiling, or the like.

In a further embodiment, at least one additional set of probes 1004 may be designed to capture regions 1002 of special interest, which may correspond to regions that contains actionable markers, such as, regions that are difficult to resolve without high coverage, regions for which haplotype resolution is important, non-coding regions containing important markers, regions important to resolve structural variants, regions corresponding to potential fusion breakpoints, and the like.

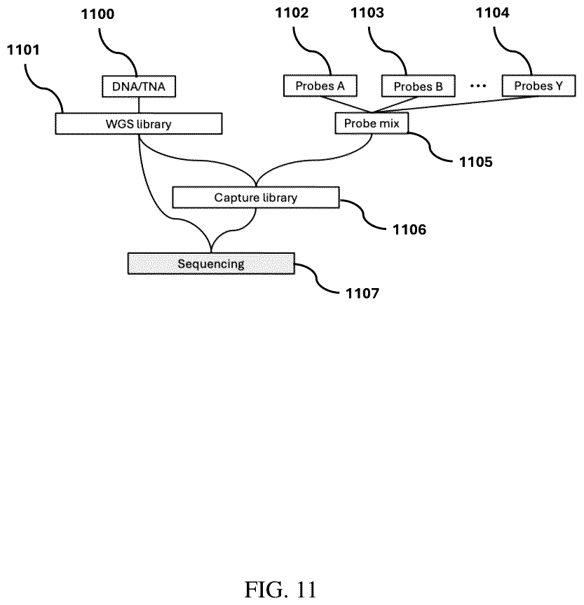

Moving to FIG. 11, a whole-genome sequencing (WGS) library 1101 may be prepared from DNA, RNA, or TNA 1100. Additionally, at least two sets of probes 1102-1104 targeting different genomic regions may be prepared and mixed in unequal proportions, so that some probes are overrepresented in the mix 1105. A capture library 1106 may be prepared from at least part of the WGS library 1101 with the probe mix 1105. Lastly, the prepared capture library 1106 may be subsequently subjected to sequencing 1107.

Further, part of, or the entirety of, the WGS library 1101 may be hybridized to the mix of target-specific capture probes 1105, to prepare a capture library 1106 that may also involve binding to streptavidin beads, extraction of DNA-bead complexes, and clean-up of the complexes. It will be apparent to those having ordinary skill in the art that variation in the target-enrichment method can be implemented.

In some embodiments, targeted enrichment may be implemented using amplicon-based enrichment. This method uses primers designed to amplify specific DNA regions of interest through PCR. In some embodiments, targeted enrichment may be implemented using hybridization capture-based enrichment. This method uses probes that are complementary to the target regions. These probes hybridize to the DNA sample, and the target regions are isolated. In some embodiments, isolation techniques include the use of biotinylated probes that bind to streptavidin beads, and/or the use of magnetic beads.

In yet a further embodiment, at least one additional set of probes may be designed to improve capture, for example by targeting alternative alleles that differ strongly from the reference genome, by adding probes to capture pseudogenes, by adding probes to decrease coverage heterogeneity, and the like.

In embodiments where different sets of probes are designed, the different sets may be mixed to obtain a single set of probes. In an additional embodiment, two distinct sets of probes, both corresponding to different sets of genomic regions of special interest, may be designed. It should be apparent to those having ordinary skill in the art that more than two sets of probes can be designed.

Moreover, different coverages may be targeted for each set of probes. For example, a given coverage may be required to identify different variants. Examples of said variants may include, single-nucleotide variants (SNVs), insertions and deletions (indels), copy number alterations (CNA), and the like.

Further, in a large array of genes, a higher coverage may be required for genomic regions that will be used to resolve complex variants, such as those occurring in regions of low complexity, those involving both substitutions and insertions/deletions, those involving highly similar paralogs, those requiring phasing of variants, and the like.

In an embodiment, a lower coverage might be desired for some set of probes, for example to counter a higher abundance of molecules in the starting material, such as in the case of mitochondrial DNA. The desired coverage for each set of regions may be a function of multiple factors, including the data generation workflow, the sequencing platform, the complexity of the region, the bioinformatic pipeline, the type of marker, and the desired limit of detection.

The desired coverage for SNVs and short indels may be predicted mathematically, for example using a binomial model to calculate the probability of observing a variant given a coverage and a variant fraction in a sample. For other markers, a first approximation of the desired coverage may be obtained by the systems and methods, based on published evidence and previous products. The required coverage may then be firmly established during development activities, using for example experiments involving in silico production of datasets with different coverages, for instance by down-sampling a large dataset, and/or producing multiple sequence datasets with different sequencing depths and potentially different starting material (e.g. dilution series).

The different sets of probes may be mixed to achieve a desired coverage for the different regions, by changing the final concentration per probe so that probes from each set are represented proportionally to the desired coverage for the regions they target, as shown in step.

As a nonlimiting example, if a first coverage (i.e., cov1) is desired for targeting one or more regions with a first set of probes (i.e., probe1) and a second coverage (i.e., cov2) is desired for targeting one or more regions with a second set of probes (i.e., probe2), the probes may be mixed such that the concentration of each probe in the second set of probes is higher than the concentration of each probe in the first set of probes by a factor equal to cov2/cov1. Alternatively, the probes may be mixed such that the concentration of each probe in the first set of probes is higher than the concentration of each probe in the second set of probes by a factor equal to cov1/cov2.

In an embodiment, if cov2 is significantly larger than cov1, the relationship between the ratio of probe concentrations and the expected ratio of coverages may not be linear, and saturation may be considered when designing the probe mix. In some embodiments, different mixes are obtained with varying proportions of the first set of probes and the second set of probes, and the whole workflow, from data generation to analysis, may be performed with each set. In such embodiments, quality metrics, coverage metrics, and metrics related to analytical and potentially technical performance, may be obtained for each of the proportions of the first set of probes and the second set of probes, and the final proportion of each set of probes is determined based on the aforementioned metrics. It will be apparent to those having ordinary skill in the art that the same approach can be used for any number of different probe sets to be mixed.

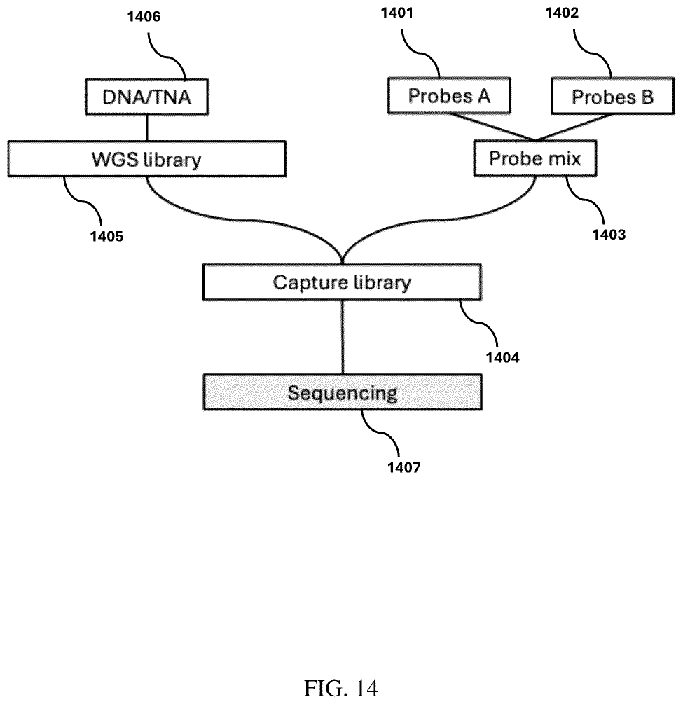

Moving on to FIG. 14, an illustration of an embodiment, where two sets of probes (i.e., Probes A 1401 and Probes B 1402) may be mixed 1403 at different concentrations before being used to generate a capture library 1404 is depicted. The capture library 1404 is generated from a WGS library 1405 derived from a DNA or TNA sample 1406 before being sequenced 1407. In an embodiment, medium coverage for a large number of genes and higher coverage for selected regions of interest may be provided.

In an embodiment, two sets of probes 1401-1402 may be designed to capture different sets of genes. To illustrate, Probes A 1401 may represent a clinical exome solution and target 6380 genes, and Probes B 1402 may represent a custom panel and target 128 genes of special clinical interest or encompassing difficult markers, such as variants representing a combination of single-nucleotide variants in highly similar paralogs.

The systems and methods may mix probes 1403 at different relative concentrations. The expected ratio of concentration of individual probes from Probes B 1402 compared to Probes A 1401 may be 0, 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, or 15. The expected ratio of concentration of individual probes from Probes A 1401 compared to Probes B 1402 may be 0, 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, or 15.

Some of the genes targeted by Probes B 1402 may also be targeted by some of Probes A 1401, although at a lower density of probes per gene, e.g., approximately 10 times, 9 times, 8 times, 7 times, 6 times, 5 times, 4 times, 3 times, or 2 times, less probes per gene in Probes A 1401 compared to Probes B 1402. Some of the genes targeted by Probes A 1401 may also be targeted by some of Probes B 1402, although at a lower density of probes per gene, e.g., approximately 10 times, 9 times, 8 times, 7 times, 6 times, 5 times, 4 times, 3 times, or 2 times, less probes per gene in Probes B 1402 compared to Probes A 1401. For those genes covered by both Probes B 1402 and Probes A 1401, the approximate expected ratio of concentration of individual probes may be 1, 1.5, 2, 2.5, 3, 3.5, 4, 4.5, 5, 5.5, 6, 6.5, 7, 7.5, 8, 8.5, 9, 9.5, 10, 11, 12, 13, 14, or 15.

In the embodiments where different sets of probes 1401-1402 were mixed 1403 at different relative concentrations, the relative representation in the capture library 1404 may differ among genomic regions.

### Methods of Generating Sequencing Libraries

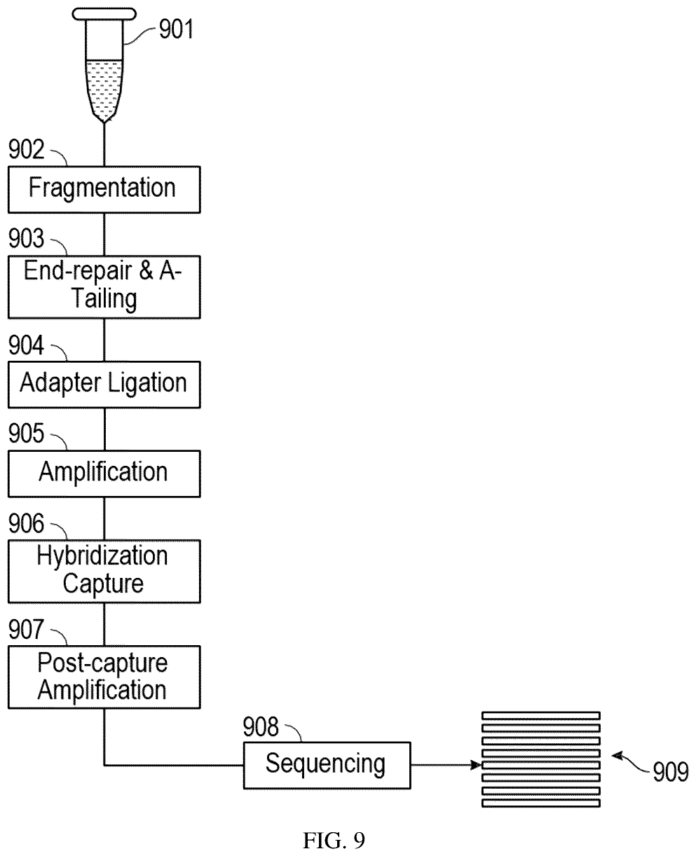

As illustrated in FIG. 9, the method of the present disclosure may include the following steps. Nucleic acid is isolated from the sample 901 of interest using a pre-existing DNA-isolation or RNA-isolation method.

DNA-isolation or DNA purification methods include, but are not limited to any known method to the skilled artisan, e.g., lysing extracted DNA from a sample using e.g., a detergent (e.g., sodium dodecyl sulphate, Triton X-100), separating the soluble DNA from the cell debris and other insoluble material, binding the DNA of interest to a purification matrix (e.g., silica), wash the bound DNA to remove impurities, and elute the bound DNA from the purification matrix. If needed, the isolated DNA is divided into multiple aliquots and each one is individually processed through steps 902-905.

RNA-isolation or RNA purification methods include, but are not limited to any known method to the skilled artisan, e.g., phenol-chloroform extraction (i.e., an extraction method that uses organic solvents to separate RNA based on the differential solubility of cellular components), column-based extraction and purification (i.e., using silica membranes or filters in a centrifuge to preferentially bind and elute RNA), magnetic bead-based extraction and purification (i.e., employing magnetic particles coated with RNA-binding surfaces to capture RNA from a solution). In some embodiments, after the RNA is extracted and purified, it is reverse transcribed into DNA (e.g., cDNA).

The nucleic acids might be isolated from a fresh or a fresh-frozen, which might be blood sample, a saliva sample, or a sample from another tissue, for example obtained via biopsy. The nucleic acids might alternatively be isolated from a formalin-fixed paraffin-embedded (FFPE) sample, for example after a tumor biopsy. In other embodiments, the nucleic acids might be cell-free DNA (cfDNA) and/or cell-free RNA (cfRNA) isolated from a bodily fluid, such as blood, urine, or cerebrospinal fluid. The cfDNA/cfRNA might contain circulating tumor DNA (ctDNA), circulating tumor RNA (ctRNA) or fetal DNA.

Referring to step 902, the DNA is optionally fragmented. Fragmentation 902 can be performed using any method known to the skilled artisan, including, but not limited to, mechanical shearing, sonication, ultrasonication, enzymatic fragmentation, partial digestion, restriction enzyme digestion. Naturally fragmented input material, such as cDNA, cfDNA, or cfRNA, may not be fragmented.

Fragmentation 902 may result in a fragmented DNA being 50 to 10000 base-pairs in length. In some embodiments, fragmentation 902 may result in fragmented DNA being 50 base-pairs to 500 base-pairs in length. In some embodiments, fragmentation 902 may result in fragmented DNA being 500 to 1000 base-pairs in length. In some embodiments, fragmentation 902 may result in fragmented DNA being 1000-2000 base-pairs in length. In some embodiments, fragmentation 902 may result in fragmented DNA being 2000-3000 base-pairs in length. In some embodiments, fragmentation 902 may result in fragmented DNA being 3000-4000 base-pairs in length. In some embodiments, fragmentation 902 may result in fragmented DNA being 4000-5000 base-pairs in length. In some embodiments, fragmentation 902 may result in fragmented DNA being 5000-6000 base-pairs in length. In some embodiments, fragmentation 902 may result in fragmented DNA being 6000-7000 base-pairs in length. In some embodiments, fragmentation 902 may result in fragmented DNA being 7000-8000 base-pairs in length. In some embodiments, fragmentation 902 may result in fragmented DNA being 8000-9000 base-pairs in length. In some embodiments, fragmentation 902 may result in fragmented DNA being 9000-10000 base-pairs in length.

The DNA fragments may derive from cfDNA, cDNA, genomic DNA, or mitochondrial DNA and may be sized-fractionated, for example by agarose gel electrophoresis; gel chromatography; equilibrium density-gradient centrifugation, including sucrose gradient centrifugation, percol gradient centrifugation, cesium-chloride centrifugation; and other means known to the skilled artisan.

Referring to step 903, the DNA fragments can be processed through end-repair and A-tailing. After fragmentation 902, the extracted DNA can be end-repaired or end-polished and a single adenine base can be added to form an overhang by an A-tailing reaction. This A-overhang allows adapters containing a single thymine base to pair with the DNA fragments.

Referring to step 904, adaptors are ligated to the ends of the DNA fragments. In some embodiments, the adapters are Y-shaped adapters. In some embodiments, the adapters include one of a diversity of molecular barcodes.

An adaptor comprises a double-stranded sequence at the end being annealed to the double-stranded DNA. In this regard, one of the two strands of the double-stranded sequences of the adaptor will be ligated to the 3′ end of the fragmented double-stranded DNA, and the other of the two strands of the double-stranded sequences of the adaptor will be ligated to the 5′ end of the fragmented double-stranded DNA.

In some embodiments, Y-shaped adapters may be ligated to fragmented DNA. The adapters may be ligated to end-repaired DNA fragments. Adapters can contain sequences required downstream or they can be extended by PCR with primers that match the ligated sequences on the 3′ end but are longer on their 5′ end to include the sequences required downstream. Among other sequences, the adapters may include sequences for indexes to allow for multiplexing, oligonucleotides that facilitate hybridization, and/or sequencing primer binding sites for forward read sequence primers and/or reverse read sequence primers to bind to the library. In some embodiments, the adapters may also include sequence identifier tags, as described subsequently.

The ends of the double-stranded sequences of the adaptors being ligated to the fragmented double-stranded DNA are not limited and may comprise blunt ends, 3′ overhangs, and 5′ overhangs. In this regard, the 5′ ends of the adaptors being ligated could either terminate with a 5′-phosphate or a 5′-OH. If a 5′-OH is at the adaptor end to be ligated to the target nucleic acid, it may be necessary to use a polynucleotide kinase to complete the backbone and join the 5′-OH of the adapter to the 3′-OH of the fragmented DNA. The DNA fragments may be repaired to create blunt ends, and an adenine (A) nucleotide may be added to the 3′ ends to prepare the DNA fragments for adapter ligation.

In some embodiments, a variable length spacer is included at the end of double-stranded sequences, using the method disclosed in WO 2021/053208. In some embodiments, the adapters contain other types of barcodes at the end of their double-stranded sequences.

Referring to step 905 of FIG. 9, DNA fragments with ligated adaptors are amplified using primers that incorporate primer-binding sites for subsequent sequencing-by-synthesis and sample-specific indexes. Amplification 905 can be performed using any known method, e.g., PCR.

In some embodiments, the result is a whole-genome sequencing library. In one embodiment, the whole-genome sequencing library is subjected to sequencing-by-synthesis 908, producing sequencing reads 909 spreads across the genome.

In some embodiments, part or all of the whole-genome sequencing library is used as input for hybridization capture, referring to step 906, using target-specific capture probes that match genomic regions of interest. DNA fragments and the hybridized matching probes are bound to streptavidin beads. The DNA-bead complexes are retrieved and cleaned up.

After release from the probes, referring to step 907, the DNA fragments are amplified using primers binding to the already incorporated primer-binding sites. In some embodiments, after clean-up, the amplified DNA fragments constitute the capture library. The capture library is subjected to sequencing-by-synthesis 908, producing sequencing reads 909.

### Differentially Tagging Different Sequencing Libraries

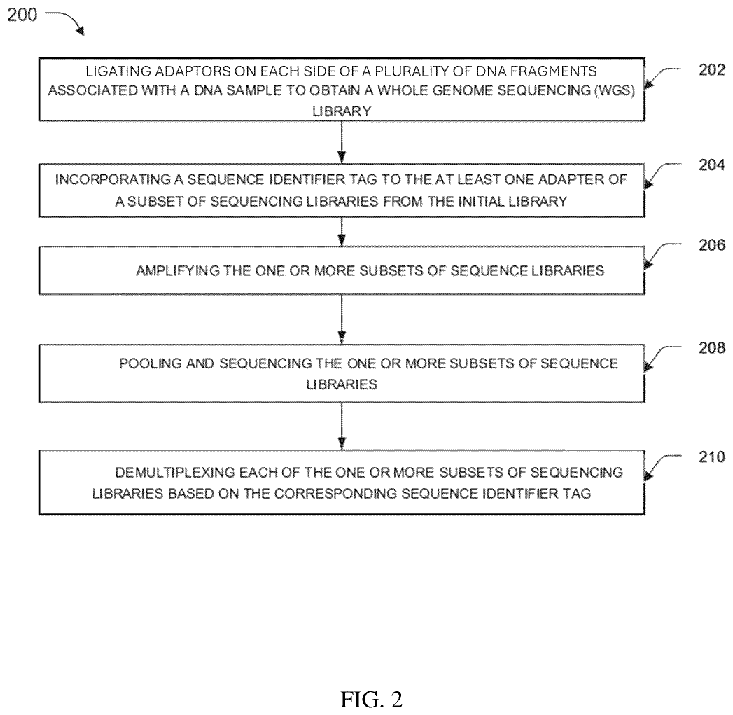

FIG. 2 illustrates a flowchart of an example method 200 for tagging multiple sequencing libraries, according to embodiments of the present disclosure. The method 200 initiates by preparing the sequencing libraries using techniques known to those skilled in the art and those described herein. At step 202, the method 200 includes ligating one adapter at each end of fragmented DNA associated with a DNA sample to obtain an initial library pool (see, also FIG. 9). In some embodiments, provided it incorporates all necessary sequences, the initial library pool may be the initial WGS library.

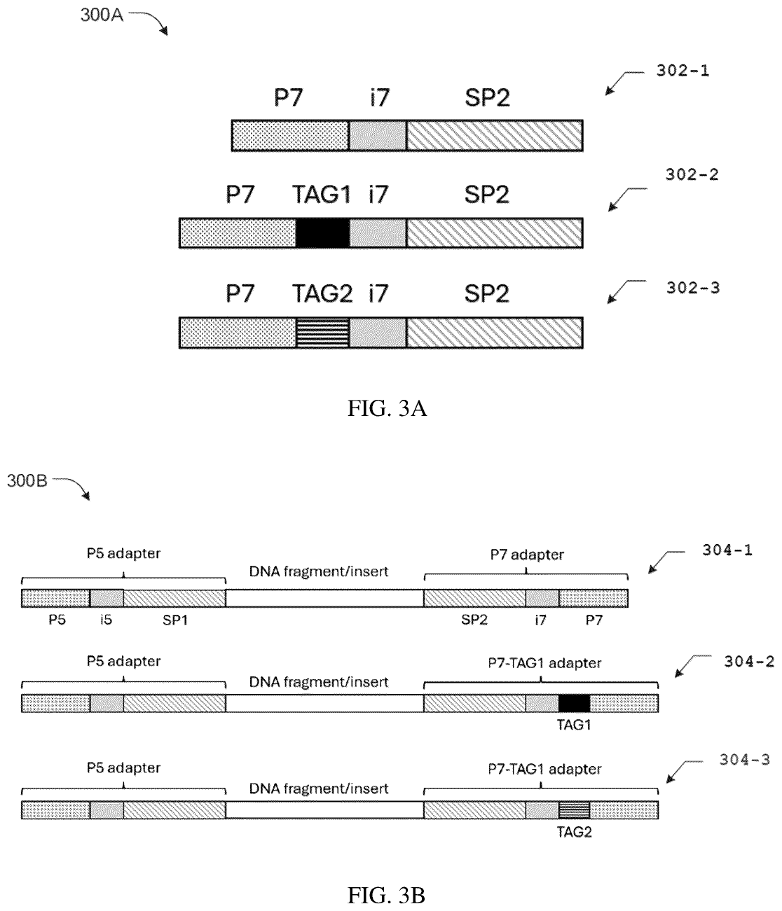

For example, representation 300A of FIG. 3A shows three example sequences for the adapter, in this case compatible with an Illumina sequencing platform. In some examples, an adapter may embody a standard sequence 302-1 indicative of a barcoded P7 adapter having a site to bind a Sequencing Primer 2, index i7, and a P7 primer-binding site. In such examples, the adapter may have no (initial) sequencing identifier tag. Alternatively, in other examples, the adapter indicated by sequence 302-2 may embody a first/initial sequence identifier tag (represented by TAG1) placed between the index sequence (i.e., i7) and at least one oligonucleotide sequence (i.e., P7). In further examples, the adapter may embody sequence 302-3, where the first sequence identifier tag TAG1 (represented by a blacked-out box) is replaced/modified to a second sequence identifier tag TAG2 (represented by a hatched box). Further, the location of the sequence identifier tags may not be limited to positions described in the foregoing examples/embodiments, and may be suitably adapted by those skilled in the art based on requirements.

In some embodiments, the first/initial sequence identifier tag TAG1 may match with the second sequence identifier tag TAG2 on 5′ end, and the first initial sequence identifier tag TAG1 may differ from the second sequence identifier tag TAG2 by at least two base pairs at 3′ end. The sequence used may be determined before incorporation. In some embodiments, the sequence identifier tags may be between approximately 3-20 base pairs in length. In some embodiments, the sequence identifier tags may be between 5-12 base pairs in length. However, the length and the location of the sequence identifier tags, and number of matches and mismatches in bases pairs between the first/initial sequence identifier TAG1 and the second sequence identifier TAG2 may be determined based on protocols executed by machines/apparatus used for sequencing the libraries.

In some embodiments, the sequence identifier tags may be 3 base pairs in length. In some embodiments, the sequence identifier tags may be 4 base pairs in length. In some embodiments, the sequence identifier tags may be 5 base pairs in length. In some embodiments, the sequence identifier tags may be 6 base pairs in length. In some embodiments, the sequence identifier tags may be 7 base pairs in length. In some embodiments, the sequence identifier tags may be 8 base pairs in length. In some embodiments, the sequence identifier tags may be 9 base pairs in length. In some embodiments, the sequence identifier tags may be 10 base pairs in length. In some embodiments, the sequence identifier tags may be 11 base pairs in length. In some embodiments, the sequence identifier tags may be 12 base pairs in length. In some embodiments, the sequence identifier tags may be 13 base pairs in length. In some embodiments, the sequence identifier tags may be 14 base pairs in length. In some embodiments, the sequence identifier tags may be 15 base pairs in length. In some embodiments, the sequence identifier tags may be 16 base pairs in length. In some embodiments, the sequence identifier tags may be 17 base pairs in length. In some embodiments, the sequence identifier tags may be 18 base pairs in length. In some embodiments, the sequence identifier tags may be 19 base pairs in length. In some embodiments, the sequence identifier tags may be 20 base pairs in length.

In some embodiments, the sequence identifier tags and/or the adapters may comprise modified nucleic acids. Non-limiting examples of modified nucleic acids comprise locked nucleic acids (LNAs), which can fine-tune sequence melting temperatures, hybridization stability, resist degradation, etc., peptide nucleic acids (PNAs), which may enhance binding affinity, resist enzyme degradation, etc., 2′-O-methyloxy-ethyl bases (2′-MOE), which may offer increased binding affinity and resist nuclease degradation, fluorobases, which have a fluorine modified ribose for increased binding affinity, 5-hydroxybutynl-2′-deoxyuridine, which is a duplex-stabilizing modified base, and 8-aza-7-deazaguanosine, which is a modified base that eliminates secondary structures associated with GC-rich sequences.

Optionally, the method 200 includes denaturing the ligated DNA fragments. Denaturing may be performed by heating the ligated DNA fragment such that the double strands thereof separate into a forward strand and a reverse strand. Further, the separated strands of the DNA fragment may be cooled to allow primers to bind to the adapters. The denatured DNA fragments (i.e., the forward and reverse strands) may be hybridized, and then amplified to create copies of the adapted DNA fragment that includes the primers/adapters. In some embodiments, a polymerase may be used for amplification, which may use polymerase chain reaction (PCR) to synthesize the corresponding strand from the denatured DNA fragment. After standard cleanup, the resulting mixture may form the initial WGS library.

Referring to FIG. 2, at step 204, the method 200 includes incorporating or modifying a sequence identifier tag to the at least one adapter of one or more subsets of sequencing libraries from the initial library pool. In embodiments where only one (or more) subset of sequencing libraries includes the sequence identifier tag, the subset of sequencing libraries may be distinguished from other sequencing libraries in the initial library pool, based on presence or absence of the sequence identifier tag.

In such embodiments, the sequencing libraries in the initial WGS library pool may have a sequence 304-1 (shown in representation 300B of FIG. 3B) including a standard barcoded P5 adapter at a first end and a standard barcoded P7 adapter at a second end. In such embodiments, at step 204, the method 200 includes incorporating a first sequence identifier TAG1. As a result, a first subset from the one or more subsets of sequencing libraries may have a sequence 304-2 including a barcoded P5 adapter, and a barcoded P7-TAG1 adapter (similar to sequence 302-2). It will be apparent to those skilled in the art that the tag may be placed in the P5 adaptor or in other adaptors suitable for different sequencing platforms.

In other embodiments, all sequencing libraries may include the first/initial sequence identifier tag TAG1. In such embodiments, at step 204, the method 200 includes modifying the first sequence identifier tag TAG1 to the second sequence identifier tag TAG2. Further, in such embodiments, the subset of sequencing libraries may be distinguished based on the corresponding sequence identifier tags.

While the foregoing examples and embodiments are described in the context of sequence identifier tags being tagged to one or two subsets of sequencing libraries, the method 200 may be suitably adapted by those skilled in the art to assign unique sequence identifier tags to any number of subsets of sequencing libraries for uniquely identifying such subsets of sequencing libraries. For example, one subset of sequencing libraries may be tagged, two subsets of sequencing libraries may be tagged, three subsets of sequencing libraries may be tagged, four subsets of sequencing libraries may be tagged, five subsets of sequencing libraries may be tagged, six subsets of sequencing libraries may be tagged, seven subsets of sequencing libraries may be tagged, eight subsets of sequencing libraries may be tagged, nine subsets of sequencing libraries may be tagged, or ten subsets of sequencing libraries may be tagged.

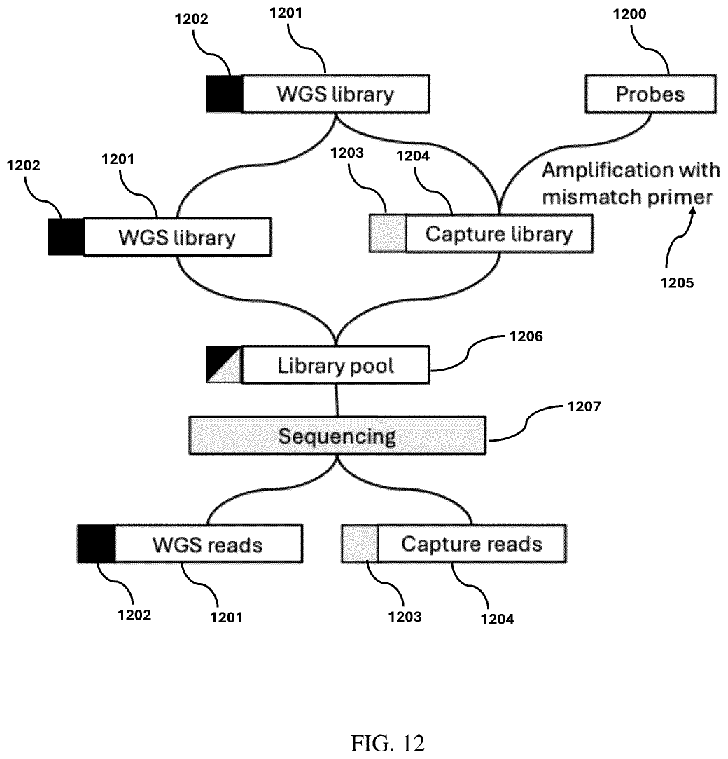

Turning to FIG. 12, an embodiment of an illustration of a workflow where different tags 1202-1203 are incorporated in the WGS library 1201 and capture library 1204, which is produced using capture probes 1200 may be shown. In such a workflow, the original WGS library 1201, which may have been a PCR-free WGS library, may be comprised of a black tag (e.g., TAG1) 1202. Such a black tag 1202 may be transformed in the capture library 1204 into a grey tag (e.g., TAG2) 1203 through primer mismatch during the post-capture PCR 1205. Each library 1201, 1204, comprising different tags 1202-1203 is subsequently pooled 1206 and sequenced 1207, resulting in the ability to differentiate one library from the other in a single NGS workflow.

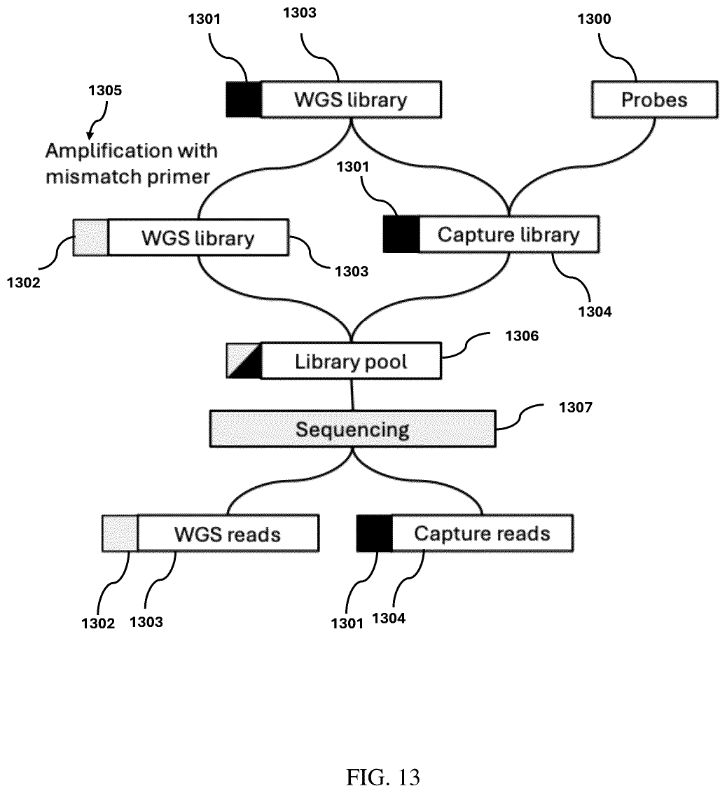

Conversely, depicted in FIG. 13 is a workflow where different tags 1301-1302 are incorporated in the WGS library 1303 and capture library 1304, which is produced using capture probes 1300. In such a workflow, the original WGS library 1303, which may or may not have been produced using PCR amplification, contains a black tag (e.g., TAG1) 1301. This black tag 1301 may be transformed into a grey tag (e.g., TAG2) 1302 through primer mismatch during a WGS PCR amplification 1305 that happens after part of the initial WGS library 1303 was used for capture. Thus, the capture library 1304 may retain the original black tag (e.g., TAG1) 1301. Each library 1303-1304, comprising different tags 1301-1302 is subsequently pooled 1306 and sequenced 1307, resulting in the ability to differentiate one library from the other in a single NGS workflow.

In some embodiments, any number of different tags may be incorporated into the libraries. For example, one tag may be incorporated into the libraries, two tags may be incorporated into the libraries, three tags may be incorporated into the libraries, four tags may be incorporated into the libraries, five tags may be incorporated into the libraries, six tags may be incorporated into the libraries, seven tags may be incorporated into the libraries, eight tags may be incorporated into the libraries, nine tags may be incorporated into the libraries, ten tags may be incorporated into the libraries, eleven tags may be incorporated into the libraries, twelve tags may be incorporated into the libraries, thirteen tags may be incorporated into the libraries, fourteen tags may be incorporated into the libraries, or fifteen tags may be incorporated into the libraries.

In an additional embodiment, two different capture libraries may be produced from the same initial WGS library, using for example two different sets of probes. In such embodiments, the TAG1 tag from the original WGS library may be transformed into TAG2 through primer mismatch during the post-capture PCR amplification of the first capture library, as described in FIG. 12. A similar process may then be used to transform the TAG1 tag from the original WGS library into a different TAG3 tag through primer mismatch during the post-capture PCR amplification of the second capture library. In said embodiment, the TAG3 tag in the second capture library may differ from the TAG2 tag in the first capture library. The two capture libraries and the initial WGS can therefore be pooled before sequencing and the reads corresponding to the WGS library, the first capture library and the second capture library can be demultiplexed based on their tag. While conducting multiple captures would incur an increase of reagent costs and handling times, such an approach might be useful in some special conditions. As a non-limiting example, one might conduct a first capture with probes targeting cDNA generated from spliced mRNA and one a second capture with probes targeting gDNA. In such a scenario, the ability to distinguish reads from the two captures would enhance the ability to assess gene expression levels and splice variation. It will be apparent to one having ordinary skill in the art that more than two different capture libraries with differing tags can be produced using a diversity of primer mismatches.

For example, two different capture libraries with differing tags can be produced using a diversity of primer mismatches, three different capture libraries with differing tags can be produced using a diversity of primer mismatches, four different capture libraries with differing tags can be produced using a diversity of primer mismatches, five different capture libraries with differing tags can be produced using a diversity of primer mismatches, six different capture libraries with differing tags can be produced using a diversity of primer mismatches, seven different capture libraries with differing tags can be produced using a diversity of primer mismatches, eight different capture libraries with differing tags can be produced using a diversity of primer mismatches, nine different capture libraries with differing tags can be produced using a diversity of primer mismatches, ten different capture libraries with differing tags can be produced using a diversity of primer mismatches, etc.

### Amplifying the Sequencing Libraries

In an embodiment, the initial WGS library may be prepared using PCR amplification. In another embodiment, the initial large panel library may be prepared using a PCR-free protocol. In the case of a PCR-free protocol, all sequences required for downstream protocol and sequencing are present in the initial adaptor. It will be apparent to those skilled in the art that the adaptor can be modified in different manners to incorporate a sequencing tag TAG1.

Referring to FIG. 2, at step 206, the method 200 includes amplifying the one or more subsets of sequencing libraries. In some embodiments, the method 200 may include amplifying only one or more of the subsets of sequencing libraries. For example, the first subset of sequencing libraries may be used for targeted enrichment using targeted sequencing techniques known to those skilled in the art. In such examples, probes specific to targeted sequences (or targeted DNA fragments) may be used for hybridization.

Streptavidin-coated beads may bind to the probes, which may be magnetically pulled and separated from other non-targeted sequences. The probes may then be eluted from the beads. Such subsets of libraries may be referred to as “capture libraries” or “enriched libraries”. The capture libraries may be subjected to PCR amplification. In other embodiments, the remaining sequencing libraries (or other subsets of sequencing libraries) may be amplified using techniques known in the art.

In an example, the capture libraries may be subjected to PCR amplification, such as using the standard P5 barcoded primer and the P7-TAG2 barcoded primer (as shown when integrated in the adapter in sequence 304-3 of FIG. 3B). The P5 and P7-TAG2 barcoded primers may be hybridized to the denatured DNA molecules and polymerases are used to create a reverse-complement copy of the DNA template.

In some embodiments, the amplification may be performed using at least one of a first polymerase lacking 3′ to 5′ exonuclease proofreading activity, and/or a second polymerase that is a proofreading polymerase. In embodiments where the first polymerase is used, the first polymerase may be active at any temperature and does not require to be heat inactivated.

In other embodiments, a mixture of two different polymerases may be used. In some embodiments, the first polymerase may be active at temperatures below a first temperature threshold and may be inactivated when the temperature exceeds a second temperature threshold. Further, the second polymerase may be active when temperature is raised above the second temperature threshold.

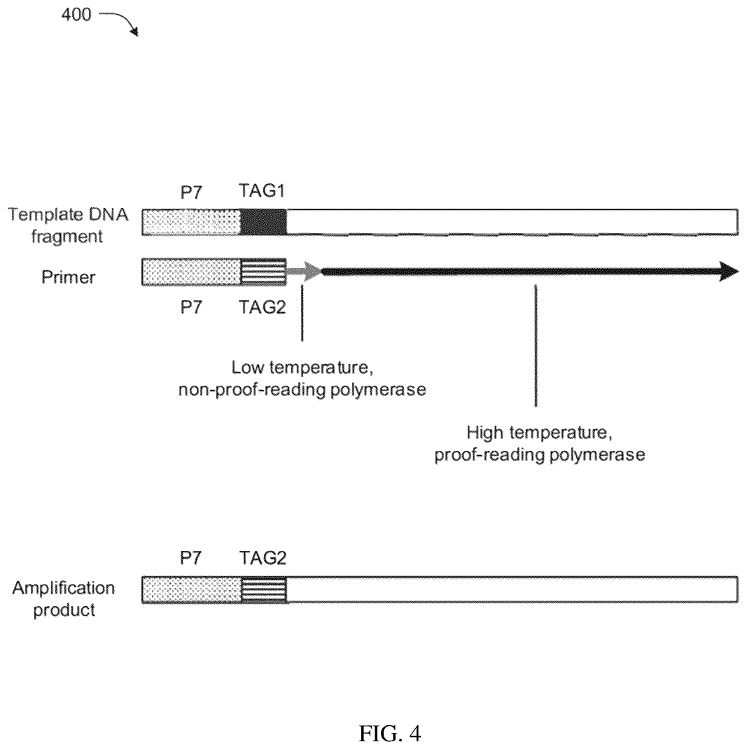

In such embodiments, the method 200 may include maintaining the temperature of template DNA fragment from the capture library pool below the first temperature threshold for a first time period (for example, 10-20 seconds at 37° C.) to activate the first polymerase, and maintaining the temperature of the template DNA fragment above the second temperature threshold for a second time period (for example, up to 75° C.) until the template DNA fragment is read to activate the second polymerase, as shown in representation 400 in FIG. 4.

As shown, when the template DNA fragment in the capture library has the first sequence tag identifier tag TAG1, and the capture library requires its sequence libraries to have the second sequence tag identifier TAG2, the first and the second polymerase may be used during amplification in turns such that portions of the template DNA fragment (which includes the adapters) are copied without error correction, while other portions are copied with error correction. For example, when the first polymerase is activated, the first polymerase may copy a portion of the template DNA fragment directly adjacent to the second sequence identifier tag TAG2 without correcting the second sequence identifier tag TAG2 to match the template DNA fragment.

Further, when the temperature of the template DNA fragment (or the solution in which the template DNA fragment is placed) exceeds the second temperature threshold, then the first polymerase is deactivated, and the second polymerase is activated. Since the second polymerase is a proofreading polymerase, the portion of the template DNA fragment away from the second sequence identifier tag TAG2 is copied faithfully to produce an amplification product (which is a copy of the template DNA sequence), with limited polymerase-induced errors. Copies of the template DNA sequence (corresponding to the sequencing library in the capture library pool) may be generated for receiving amplified signals during sequencing.

In other embodiments, the first polymerase may be used only for post-capture amplification. Since error rates of most polymerase that lack of 3′ to 5′ exonuclease activity is about 2×10⁻⁴, the use of a non-proofreading polymerase is therefore a viable option in some cases.

In further embodiments, the secondary amplification (where the sequence identifier tags may be modified) can be performed by the first polymerase, and the ensuing error rate may be acceptable for most applications. For example, low-pass WGS is typically used for the detection of large copy number alterations or genomic rearrangements, which is not impacted by an elevated error rate of the first polymerase. In such embodiments, the capture library pool is amplified with a proofreading polymerase, as typically used in NGS assays, limiting its error rate.

After post-amplification clean-up, the capture library pool may include DNA fragments mostly from the targeted genomic regions, that incorporate the second sequence identifier tag TAG2 (or any other tag that allow it to be distinguished from other sequencing libraries in the initial WGS library pool) between the P7 and i7 portions. It will be apparent to those skilled in the art that the tag TAG2 may be inserted in different locations with a different adapter design.

### Pooling and Sequencing the Sequencing Libraries

Furthermore, part of the cleaned-up WGS library may be mixed with the capture library, so that all genomic regions are represented in the final library, although at a generally lower concentration for regions not targeted by the capture probes. Moreover, before pooling the WGS and capture libraries, the TAG1 tag from the original WGS library may be transformed into a TAG2 tag through primer mismatch during a PCR amplification conducted after the part of the initial WGS library was taken to produce the capture library. As a nonlimiting example, a WGS library may incorporate a TAG2 tag, while the capture library may incorporate the original TAG1 or another tag if a modification happened after the target enrichment.

It will be apparent to one having ordinary skill in the art that other types of NGS libraries, such as those based on multiplex PCR (amplicon), rolling-circle amplification, molecular-inversion probe enrichment, tagmentation, and the like, can be combined with the WGS library and possibly one or more libraries based on hybrid capture.

Referring to FIG. 2, at step 208, the method 200 includes pooling and sequencing the one or more subsets of sequencing libraries. The sequencing libraries may be sequenced using techniques known to those skilled in the art, such as sequencing-by-synthesis, or sequencing-by-ligation, and the like. At step 210, the method 200 includes demultiplexing each of the one or more subsets of sequencing libraries based on the corresponding sequence identifier tag. Since the sequencing libraries are distinguishable using the sequence identifier tags, the demultiplexing may be performed based on the corresponding sequence identifier tags of each sequence library. The sequence libraries may accordingly be stored and analyzed based on their corresponding sequence identifier tags.

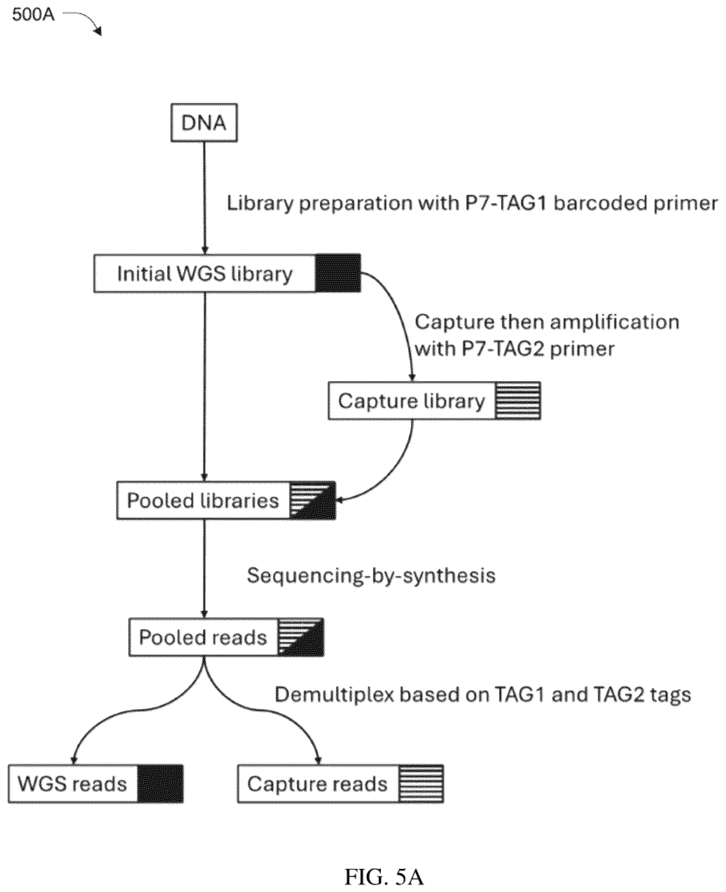

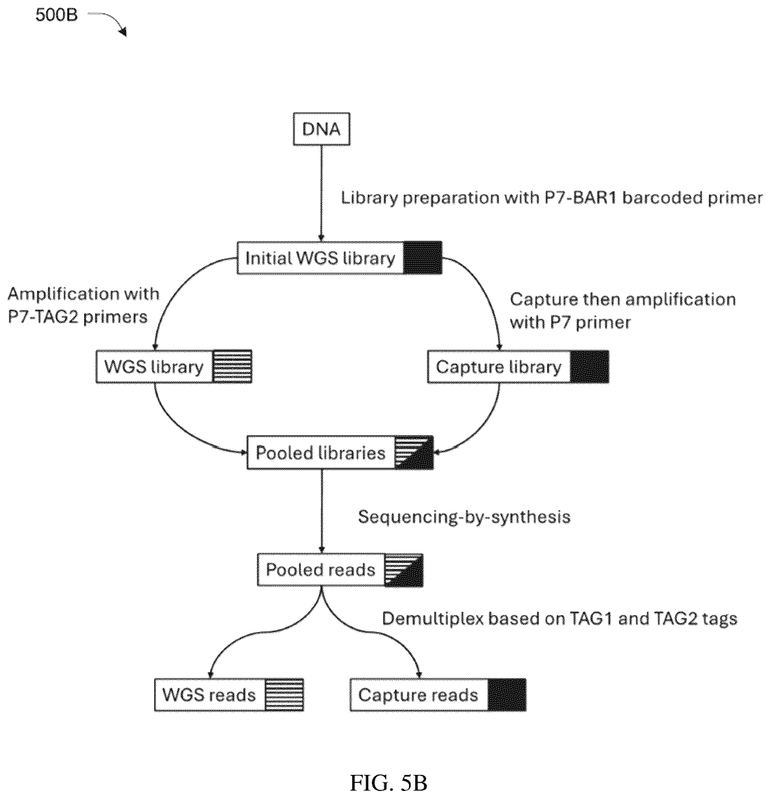

In some embodiments, any combination of sequence identifier tags may be associated with any of the sequencing libraries. For example, when the initial WGS library pool has either no tag or the first sequence identifier tag TAG1 associated therewith, a different sequence identifier tag (such as second sequence identifier tag TAG2) may be attached to the subset of sequencing libraries to be used for targeting sequencing/enrichment. The sequence libraries (the WGS library pool and the capture library pool) may then be pooled, sequenced, and demultiplexed based on their corresponding sequence identifier tags, as shown in flow diagram 500A of FIG. 5A.

In other examples, the first sequence identifier tag TAG1 may be retained for the subset of sequencing libraries to be used for targeting sequencing/enrichment, while the sequence identifier tag for the remaining sequencing libraries associated with the initial WGS library pool may be modified to the second sequence identifier tag TAG2. The sequence libraries (the capture library pool and the initial WGS library pool) may then be pooled, sequenced, and demultiplexed based on their corresponding sequence identifier tags, as shown in flow diagram 500B of FIG. 5B.

In an embodiment, the prepared library, which may contain the target-enrichment library, may be obtained with a mixture of different sets of probes. In another embodiment, the WGS library, may be subjected to high-throughput sequencing. For example, the high throughput sequencing may include sequencing-by-synthesis, sequencing-by-ligation, or the like.

Sequencing reads may subsequently be subjected to filtering and trimming, which removes: (1) low-quality reads; (2) low-quality parts of reads; (3) adapters, and the like. In a further embodiment, differing tags (e.g. TAG1 and TAG2) may be incorporated into the WGS and capture libraries, reads originating from these libraries may be demultiplexed based on the presence of the tags. In an additional embodiment, differing tags (e.g., TAG2 and TAG3) may be incorporated in two distinct capture libraries that were pooled, wherein reads originating from each of these libraries are demultiplexed based on the presence of the tags.

### Processing Sequencing Data

In yet a further embodiment, the systems and methods may include tools for processing data. For instance, said processing tools may include, bioinformatic pipelines adapted to allow analyses across all covered genomic regions or restricted to those with a given targeted coverage, corresponding to a given set of probes, or originating from different steps during the data generation workflow.

The cleaned sequencing reads may be aligned to a reference genome, using methods known to those having ordinary skill in the art.

All aligned reads may be used to infer the presence of genetic variants, such as single nucleotide variants (SNVs), insertions and deletions (indels), copy number alterations, or other variants. Further, reads from at least one of the categories of expected coverage may be treated independently, allowing a selection of filters, thresholds, and algorithms for SNV and indel detection tailored for each coverage value.

In another embodiment, reads corresponding to one set of targets, for example those for which higher coverage is expected, may be analyzed separately. In addition to SNVs and indels, said reads may be used to infer other types of genetic variants, such as copy number alterations at the infra-gene level, gene fusions, and the like.

In some embodiments, the aforementioned reads may be used to resolve allelic haplotypes, for example to identify star alleles. Moreover, reads corresponding to the set of targets for which high coverage is expected may be used to analyze error-prone regions, such as those associated to mononucleotide repeats or others short repeats. Additionally, starting with RNA, reads corresponding to the set of targets for which high coverage is expected may be used to infer changes in gene expression levels and/or splice variants.

In an embodiment, reads corresponding to one category of coverage, such as lower coverage from the WGS library outside of the regions targeted by the probes, may be extracted and used to infer genome-wide patterns, for example via the calculation of a genomic instability index. In another embodiment, WGS and capture libraries may have differing tags, thus all reads originating in the whole-genome sequencing library may be used to infer such genome-wide patterns.

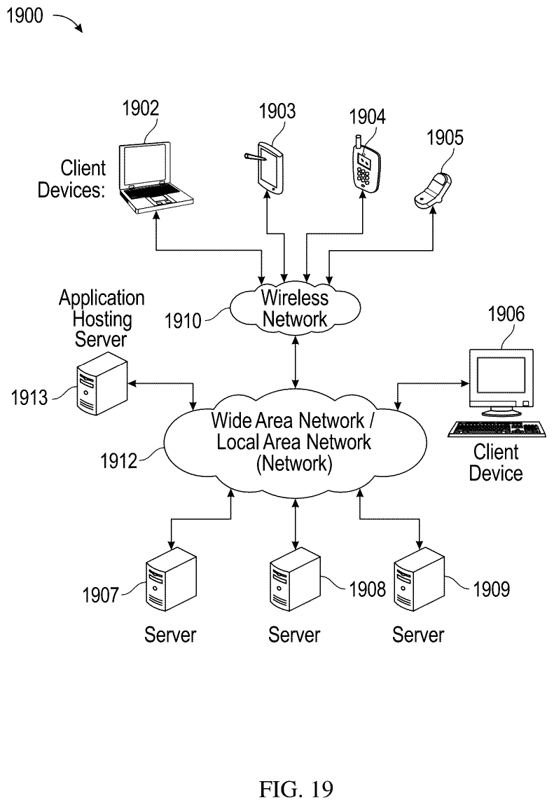

FIG. 19 illustrates components of one embodiment of an environment in which the present disclosure may be practiced. Not all of the components may be required to practice the present disclosure, and variations in the arrangement and type of the components may be made without departing from the spirit or scope of the present disclosure. As shown, the system 1900 includes one or more Local Area Networks (“LANs”)/Wide Area Networks (“WANs”) 1912, one or more wireless networks 1910, one or more wired or wireless client devices 1906, mobile or other wireless client devices 1902-1905, servers 1907-1909, and may include or communicate with one or more data stores or databases. The client devices 1902-1906 may include, for example, at least one of desktop computers, laptop computers, set top boxes, tablets, cell phones, smart phones, smart speakers, wearable devices (such as the Apple Watch) and the like. Servers 1907-1909 can include, for example, one or more application servers, content servers, search servers, and the like. FIG. 19 also illustrates application hosting server 1913.

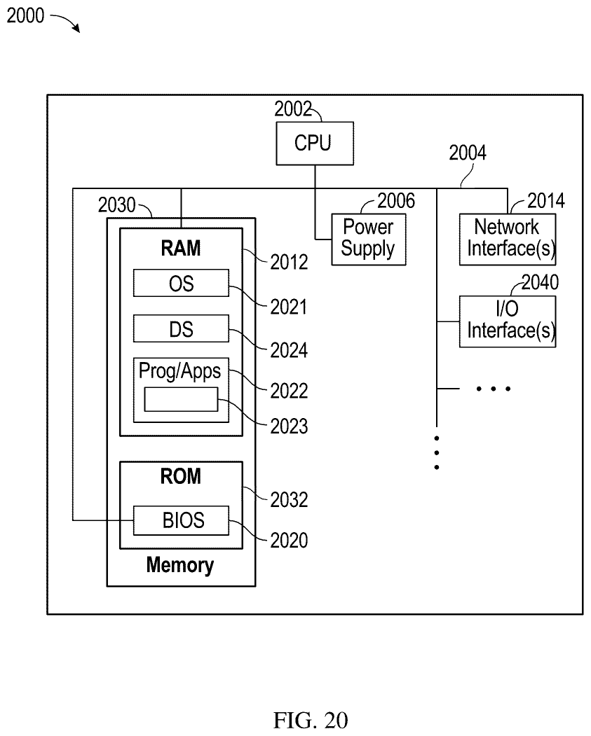

FIG. 20 illustrates a block diagram of an electronic device 2000 that can implement one or more aspects of an apparatus, system, and method for measurement and secure transmission of physical properties (the “Engine”) according to one embodiment of the present disclosure. Instances of the electronic device 2000 may include servers, e.g., servers 1907-1909, and client devices, e.g., client devices 1902-1906. In general, the electronic device 2000 can include a processor/CPU 2002, memory 2030, a power supply 2006, and input/output (I/O) components/devices 2040, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 2000. A touchscreen may determine whether a user is providing input by, for example, determining whether the user is touching the touchscreen with a part of the user's body such as his or her fingers. The electronic device 2000 can also include a communications bus 2004 that connects the aforementioned elements of the electronic device 2000. Network interfaces 2014 can include a receiver and a transmitter (or transceiver), and one or more antennas for wireless communications.

The processor 2002 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 2030, which can include Random Access Memory (RAM) 2012 and Read Only Memory (ROM) 2032, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 2021, data storage 2024, which may include one or more databases, and programs and/or applications 2022, which can include, for example, software aspects of the program 2023. The ROM 2032 can also include Basic Input/Output System (BIOS) 2020 of the electronic device.

Software aspects of the program 2023 are intended to broadly include or represent all programming, applications, algorithms, models, software, and other tools necessary to implement or facilitate methods and systems according to embodiments of the present disclosure. The elements may exist on a single computer or be distributed among multiple computers, servers, devices, or entities.

The power supply 2006 contains one or more power components and facilitates supply and management of power to the electronic device 2000.

The input/output components, including Input/Output (I/O) interfaces 2040, can include, for example, any interfaces for facilitating communication between any components of the electronic device 2000, components of external devices (e.g., components of other devices of the network or system 1900), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 2040 and the bus 2004 can facilitate communication between components of the electronic device 2000, and in an example can case processing performed by the processor 2002.

Where the electronic device 2000 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system, and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 1902-1906, as may be used in an example apparatus, system and method embodying the Engine, may range widely in terms of capabilities and features. For example, a cell phone, smart phone, or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 1902-1906, for example, as may be used in an example apparatus, system and method implementing the Engine, may run a variety of operating systems, including personal computer operating systems such as Windows, iOS or Linux, and mobile operating systems such as IOS, Android, Windows Mobile, and the like.

Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 1910 or 1912, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 1910, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPV6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present disclosure include apparatuses, systems, and methods implementing the Engine. Embodiments of the present disclosure may be implemented on one or more of client devices 1902-1906, which are communicatively coupled to servers including servers 1907-1909. Moreover, client devices 1902-1906 may be communicatively (wirelessly or wired) coupled to one another. In particular, software aspects of the Engine may be implemented in the program 2023. The program 2023 may be implemented on one or more client devices 1902-1906, one or more servers 1907-1909, and 1913, or a combination of one or more client devices 1902-1906, and one or more servers 1907-1909 and 1913.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

The method may be carried out by a software device located on a cloud computing server to permit decentralized analysis. In an embodiment, the cloud computing server may comprise a global center that provides central services, such as user authentication and authorization. In one embodiment, the cloud computing server may comprise at least one regional center to provide file management, storage, and other functionalities. It is contemplated that this permits the users to access the software from a server that complies with local requirements and regulations.

### Practical Applications

The systems and methods as described herein disclose a process to produce a NGS assay that targets different genomic regions at different depths and granularity. In an embodiment, the systems and methods, may be utilized with large panels with increased resolution of some regions germline analysis. For example, the systems and methods may be deployed to produce a NGS assay targeting multiple genes, such as a clinical exome solution, that can be adopted to identify and diagnose a large array of hereditary diseases, while offering increased resolution of some regions known to contain important markers that are difficult to resolve, alternative alleles that are not efficiently captured by probes based on the reference genome, important non-coding markers, or other desired features. Such an assay will be obtained by combining probes for the large panel, with probes for increased resolution of specific regions.

In another embodiment, the systems and methods may be utilized with large panels with increased resolution of some regions. The systems and methods may be deployed to produce a NGS assay targeting multiple genes, such as a comprehensive genomic profiling panel, that can be adopted to identify, diagnose, and monitor somatic diseases, such as cancer, while offering increased resolution of some regions. Such an assay may be obtained by combining probes for a large panel, with probes for increased resolution of specific regions. Probes B may be designed to resolve complex variants, such as those involving a combination of substitutions and insertions/deletions, those in low-diversity regions, such as mono or di-nucleotide repeats, those involving gene fusions, and the like. In an additional embodiment, parts or the whole of Probes B may be designed to cover markers of interest in non-coding regions. As a nonlimiting example, Probes B may be designed to target microsatellite loci, which are then used to compute a microsatellite instability score. As another nonlimiting example, the capture library may be based on a combination of Probes A and Probes B, and also may be complemented by part of the WGS library. The WGS reads, potentially identified based on tags modified either in the WGS or in the capture library, may be used to infer genome-wide patterns, such as a genomic instability index.

In yet a further embodiment, the systems and methods may include a whole-genome sequencing solution with increased resolution of some regions. The systems and methods may be exploited to produce a NGS assay capable of providing a complete representation of the genome, but with increased representation of some regions that are of special interest and difficult to resolve without higher coverage. In such an embodiment, the NGS assay may be achieved by combining the original WGS library, which can be obtained using a PCR-free method, with a capture library, which may be obtained based on a mix of different sets of probes or a single probe set. The probes set will typically target regions where identification of complex variants, such as those involving a combination of substitutions and insertions/deletions, those in low-diversity regions, such as mono or di-nucleotide repeats, those involving gene fusions, and the like, are important or regions where allele phasing is important, for example to identify star alleles. It will be apparent to those having ordinary skill in the art that such an assay may be adapted for germline or somatic contexts, by selecting appropriate sets of probes, and that an assay suitable for both contexts can be obtained by combining sets of probes.

Additionally, targeted assays with recognizable low-coverage WGS for somatic contexts may be utilized by the systems and methods. In an embodiment, the systems and methods may be deployed to produce a NGS assay that targets genes of clinical importance for somatic diseases, such as cancer, while simultaneously providing a low-coverage scan of the whole genome that can be used to infer genome-wide patterns, such as a genomic instability index. In such embodiments, the WGS and capture libraries can be tagged differently, so that reads originating from each can be identified from the sequencing outputs and analyzed distinctively.

In some embodiments, target genomic regions belong to genes associated with any disease or condition. In some embodiments, target genomic regions belong to any gene of interest.

In some embodiments, target genomic regions belong to genes associated with cancer. Cancer types can be grouped into broader categories. The main categories of cancer include: carcinoma (meaning a cancer that begins in the skin or in tissues that line or cover internal organs, and its subtypes, including adenocarcinoma, basal cell carcinoma, squamous cell carcinoma, and transitional cell carcinoma); sarcoma (meaning a cancer that begins in bone, cartilage, fat, muscle, blood vessels, or other connective or supportive tissue); leukemia (meaning a cancer that starts in blood-forming tissue (e.g., bone marrow) and causes large numbers of abnormal blood cells to be produced and enter the blood; lymphoma and myeloma (meaning cancers that begin in the cells of the immune system); and central nervous system cancers (meaning cancers that begin in the tissues of the brain and spinal cord).

Examples of carcinomas include, without limitation, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, an adenocarcinoma, a gastrinoma, a cholangiocarcinoma, a hepatocellular carcinoma, a combined hepatocellular carcinoma and cholangiocarcinoma, a trabecular adenocarcinoma, an adenoid cystic carcinoma, an adenocarcinoma in adenomatous polyp, an adenocarcinoma, familial polyposis coli, a solid carcinoma, a carcinoid tumor, a branchiolo-alveolar adenocarcinoma, a papillary adenocarcinoma, a chromophobe carcinoma, an acidophil carcinoma, an oxyphilic adenocarcinoma, a basophil carcinoma, a clear cell adenocarcinoma, a granular cell carcinoma, a follicular adenocarcinoma, a non-encapsulating sclerosing carcinoma, adrenal cortical carcinoma, an endometroid carcinoma, a skin appendage carcinoma, an apocrine adenocarcinoma, a sebaceous adenocarcinoma, a ceruminous adenocarcinoma, a mucoepidermoid carcinoma, a cystadenocarcinoma, a papillary cystadenocarcinoma, a papillary serous cystadenocarcinoma, a mucinous cystadenocarcinoma, a mucinous adenocarcinoma, a signet ring cell carcinoma, an infiltrating duct carcinoma, a medullary carcinoma, a lobular carcinoma, an inflammatory carcinoma, Paget's disease, a mammary acinar cell carcinoma, an adenosquamous carcinoma, an adenocarcinoma w/squamous metaplasia, a sertoli cell carcinoma, embryonal carcinoma, choriocarcinoma.

Examples of sarcomas include, without limitation, glomangiosarcoma, sarcoma, fibrosarcoma, myxosarcoma, liposarcoma, leiomyosarcoma, rhabdomyosarcoma, embryonal rhabdomyosarcoma, alveolar rhabdomyosarcoma, stromal sarcoma, carcinosarcoma, synovial sarcoma, hemangiosarcoma, kaposi's sarcoma, lymphangiosarcoma, osteosarcoma, juxtacortical osteosarcoma, chondrosarcoma, mesenchymal chondrosarcoma, giant cell tumor of bone, ewing's sarcoma, odontogenic tumor, malignant, ameloblastic odontosarcoma, ameloblastoma, malignant, ameloblastic fibrosarcoma, myeloid sarcoma, mast cell sarcoma.

Examples of leukemias include, without limitation, leukemia, lymphoid leukemia, plasma cell leukemia, erythroleukemia, lymphosarcoma cell leukemia, myeloid leukemia, basophilic leukemia, cosinophilic leukemia, monocytic leukemia, mast cell leukemia, megakaryoblastic leukemia, and hairy cell leukemia.

Examples of lymphomas and myelomas include, without limitation, malignant lymphoma, Hodgkin's disease, Hodgkin's, paragranuloma, malignant lymphoma, small lymphocytic, malignant lymphoma, large cell, diffuse, malignant lymphoma, follicular, mycosis fungoides, other specified non-Hodgkin lymphomas, myeloma, and multiple myeloma.

Examples of melanomas include, without limitation, malignant melanoma, amelanotic melanoma, superficial spreading melanoma, malignant melanoma in giant pigmented nevus, and epithelioid cell melanoma.

Examples of brain/spinal cord cancers include, without limitation, pincaloma, malignant, chordoma, glioma, malignant, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, malignant, neurofibrosarcoma, neurilemmoma, malignant.

Examples of other cancers include, without limitation, a thymoma, an ovarian stromal tumor, a the coma, a granulosa cell tumor, an androblastoma, a leydig cell tumor, a lipid cell tumor, a paraganglioma, an extra-mammary paraganglioma, a pheochromocytoma, blue nevus, malignant, fibrous histiocytoma, malignant, mixed tumor, malignant, mullerian mixed tumor, nephroblastoma, hepatoblastoma, mesenchymoma, malignant, brenner tumor, malignant, phyllodes tumor, malignant, mesothelioma, malignant, dysgerminoma, teratoma, malignant, struma ovarii, malignant, mesonephroma, malignant, hemangioendothelioma, malignant, hemangiopericytoma, malignant, chondroblastoma, malignant, granular cell tumor, malignant, malignant histiocytosis, immunoproliferative small intestinal disease.

In some embodiments, target genomic regions belong to genes associated with autoimmune diseases, including but not limited to acquired hemophilia, acromegaly, agammaglobulinemia, alopecia arcata, amyloidosis, ankylosing spondylitis, antiphospholipid syndrome, aplastic anemia, arteriosclerosis, Addison's disease, celiac disease, chagas disease, chronic autoimmune urticaria, Churg-Strauss syndrome, Cogan's disease, Crohn's disease, dermatitis herpetiformis, discoid lupus, eczema, endometriosis, cosinophilic esophagitis, cosinophilic fasciitis, Evans syndrome, giant cell myocarditis, giant cell arteritis, Graves' disease, Guillian-Barre syndrome, Hashimoto's thyroiditis, interstitial cystitis, Kawasaki disease, lupus, Lyme disease, mixed connective tissue disease, multiple sclerosis, narcolepsy, palindromic rheumatism, polymyalgia rheumatica, polymyositis, primary biliary cirrhosis, psoriasis, psoriatic arthritis, Raynaud's syndrome, reactive arthritis, rheumatic fever, rheumatoid arthritis, sarcoidosis, scleritis, Sjogren's syndrome, small fiber sensory neuropathy, Takayasu arthritis, testicular autoimmunity, type 1 diabetes, ulcerative colitis, undifferentiated connective tissue disease, and vitiligo.

In some embodiments, target genomic regions belong to genes associated with cardiovascular disease, including, but not limited to, heart failure, peripheral artery disease, coronary artery disease, arrhythmia, hypertension, congenital heart disease, cardiomyopathy, valvular heart disease, aortic aneurysm, deep vein thrombosis, pulmonary embolism, myocarditis, pericarditis, and rheumatic heart disease.

In some embodiments, target genomic regions belong to genes associated with neurological disease including, but not limited to, Parkinson's disease, spinal cord injury, stroke, Alzheimer's disease, amyotrophic lateral sclerosis, multiple sclerosis, epilepsy, migraine, Huntington's disease, peripheral neuropathy, traumatic brain injury, cerebral palsy, autism spectrum disorder, Tourette syndrome, dementia, meningitis, and neurofibromatosis.

In some embodiments, target genomic regions belong to genes associated with other diseases or conditions, including but not limited to, liver failure, kidney disease, sickle cell anemia, beta-thalassemia, muscular dystrophy, muscular atrophy, progeria, Wilson disease, Gaucher disease, Pompe disease, Rett syndrome, Ehlers-Danlos syndrome, Marfan syndrome, idiopathic pulmonary fibrosis (IPF), and amyloidosis.

### EXAMPLES

Different experiments were conducted to establish the functionality of the present disclosure. The experimental data and examples provided in this disclosure are intended solely to illustrate the methods described and should not be construed as limiting the scope of the invention. These examples are presented to enhance the understanding of the invention, offering clarity without restricting the full breadth of the disclosed methods.

### Example 1: Conversion of Capture Library Pool Using a Combination of Non-Proofreading Polymerases and Proofreading Polymerases

In a first example, conversion of capture library pool using a combination of non-proofreading polymerases and proofreading polymerases was tested.

Two reference samples (S1 and S2) were used to generate an initial WGS library and the capture library pool was produced using the method 200, with a combination of proofreading (HiFi Polymerase) and non-proofreading (Klenow Fragment) polymerases to amplify the capture library pool using a standard P5 primer and two different versions of P7-TAG2 primers, with matching P7-TAG1 sequences:

Standard P5 and P7 primer sequences:

P5
(SEQ ID NO: 21)
5′ AATGATACGGCGACCACCGAGATCTACAC 3′
P7
(SEQ ID NO: 22)
5′ CAAGCAGAAGACGGCATACGAGAT 3′

First pair of the reference samples include:

P7-TAG1a
(SEQ ID NO: 1)
5′ CAAGCAGAAGACGGCATACGAGATTAGCG 3′
P7-TAG2a
(SEQ ID NO: 2)
5′ CAAGCAGAAGACGGCATACGAGATCGGCG 3′

Second pair of reference samples include:

P7-TAG1b
(SEQ ID NO: 3)
5′ CAAGCAGAAGACGGCATACGAGATGCGCG 3′
P7-TAG2b
(SEQ ID NO: 4)
5′ CAAGCAGAAGACGGCATACGAGATTAGCG 3′

For each reference sample, the two pairs of tags were used, with different sample indexes, resulting in eight library preparations. Each of the library preparations include a WGS and a capture-targeted child. The pooled, barcoded libraries were sequenced on an Illumina® Miseq V3 flowcell, producing between 2.5 and 6.3 million reads per sample (paired-end 150 base pairs). The sequencing reads were demultiplexed per sample, using the 8 base pair sample barcodes along with the 5 base pair sequence identifier tags (such as first or second sequence identifier tags TAG1, TAG2), resulting in 16 individual barcode sequences. The percentage of reads within each category was used to monitor the success of the method of the present disclosure.

TABLE 1
Sample Barcodes Used for Demultiplexing of 8
Samples with 8 base pair Sample Barcodes in
i5 and i7 (which can be Unique or Combina-
torial Dual Indices) and 5 base pairs for
Sequence Identifier Tags (TAG1/TAG2).

| | | i7 index | | |
|---|---|---|---|---|
| | | Sequence + | | |
| | | TAG Se- | i5_ | i15 |
| | i7_In- | quence | Index_ | index2 |
| Sample | dex_ID | (bold) | ID | Sequence |
| S1- | i7_ | TCCTTGGT | i5_ | CCGTTACT |
| TAG1a- | sgUN49C | CGCCG | sgUN49 | |
| 1-C | | (SEQ ID | | |
| | | NO: 5) | | |
| S1- | i7_ | TCCTTGGT | i5_ | TAGCGACT |
| TAG1b- | sgUN49C | GCGCG | sgUN50 | |
| 1-C | | (SEQ ID | | |
| | | NO: 6) | | |
| S1- | i7_ | GTTGCTCG | i5_ | CAGTAGTC |
| TAG1a- | sgUN51C | CGCCG | sgUN51 | |
| 2-C | | (SEQ ID | | |
| | | NO: 7) | | |
| S1- | i7_ | TCCTTGGT | i5_ | ATCTGGTG |
| TAG1b- | sgUN49C | GCGCG | sgUN52 | |
| 2-C | | (SEQ ID | | |
| | | NO: 8) | | |
| S2- | i7_ | GTTGCTCG | i5_ | CTAACTAC |
| TAG1a- | sgUN51C | CGCCG | sgUN53 | |
| 1-C | | (SEQ ID | | |
| | | NO: 9) | | |
| S2- | i7_ | AGTCAAGA | i5_ | ATTCAGCT |
| TAG1b- | sgUN52C | GCGCG | sgUN54 | |
| 1-C | | (SEQ ID | | |
| | | NO: 10) | | |
| S2- | i7_ | AGTCAAGA | i5_ | TACCAGGC |
| TAG1a- | sgUN52C | CGCCG | sgUN55 | |
| 2-C | | (SEQ ID | | |
| | | NO: 11) | | |
| S2- | i7_ | AGTCAAGA | i5_ | ACCGATAA |
| TAG1b- | sgUN52C | GCGCG | sgUN56 | |
| 2-C | | (SEQ ID | | |
| | | NO: 12) | | |
| S1- | i7_ | TCCTTGGT | i5_ | CCGTTACT |
| TAG2a- | sgUN49W | CGCTA | sgUN49 | |
| 1-W | | (SEQ ID | | |
| | | NO: 13) | | |
| S1- | i7_ | TCCTTGGT | i5_ | TAGCGACT |
| TAG2b- | sgUN49W | CGCAT | sgUN50 | |
| 1-W | | (SEQ ID | | |
| | | NO: 14) | | |
| S1- | i7_ | GTTGCTCG | i5_ | CAGTAGTC |
| TAG2a- | sgUN51W | CGCTA | sgUN51 | |
| 2-W | | (SEQ ID | | |
| | | NO: 15) | | |
| S1- | i7_ | TCCTTGGT | i5_ | ATCTGGTG |
| TAG2b- | sgUN49W | CGCAT | sgUN52 | |
| 2-W | | (SEQ ID | | |
| | | NO: 16) | | |
| S2- | i7_ | GTTGCTCG | i5_ | CTAACTAC |
| TAG2a- | sgUN51W | CGCTA | sgUN53 | |
| 1-W | | (SEQ ID | | |
| | | NO: 17) | | |
| S2- | i7_ | AGTCAAGA | i5_ | ATTCAGCT |
| TAG2b- | sgUN52W | CGCAT | sgUN54 | |
| 1-W | | (SEQ ID | | |
| | | NO: 18) | | |
| S2- | i7_ | AGTCAAGA | i5_ | TACCAGGC |
| TAG2a- | sgUN52W | CGCTA | sgUN55 | |
| 2-W | | (SEQ ID | | |
| | | NO: 19) | | |
| S2- | i7_ | AGTCAAGA | i5_ | ACCGATAA |
| TAG2b- | sgUN52W | CGCAT | sgUN56 | |
| 2-W | | (SEQ ID | | |
| | | NO: 20) | | |

TABLE 2
Number a reads per library after demultiplexing
with 8 base pair sample barcodes along with 5
base pair sequence identifier tags (TAG1/TAG2).

| Sample ID | Number of reads | Number of mapped reads |
|---|---|---|
| S1-TAG2a-1-C | 6,329,054 | 6,199,622 |
| S1-TAG1a-1-W | 185,006 | 181,278 |
| S1-TAG2a-2-C | 4,950,936 | 4,850,484 |
| S1-TAG1a-2-W | 180,834 | 177,320 |
| S1-TAG2b-1-C | 6,725,070 | 6,589,444 |
| S1-TAG1b-1-W | 169,072 | 165,798 |
| S1-TAG2b-2-C | 8,746,808 | 8,569,788 |
| S1-TAG1b-2-W | 215,498 | 211,140 |
| S2-TAG2a-1-C | 3,430,376 | 3,359,268 |
| S2-TAG1a-1-W | 128,180 | 125,524 |
| S2-TAG2a-2-C | 2,501,536 | 2,450,854 |
| S2-TAG1a-2-W | 112,008 | 109,636 |
| S2-TAG2b-1-C | 4,618,102 | 4,525,468 |
| S2-TAG1b-1-W | 61,372 | 60,116 |
| S2-TAG2b-2-C | 4,650,980 | 4,557,606 |
| S2-TAG1b-2-W | 62,186 | 60,846 |
| Total | 43,067,018 | 42,194,192 |

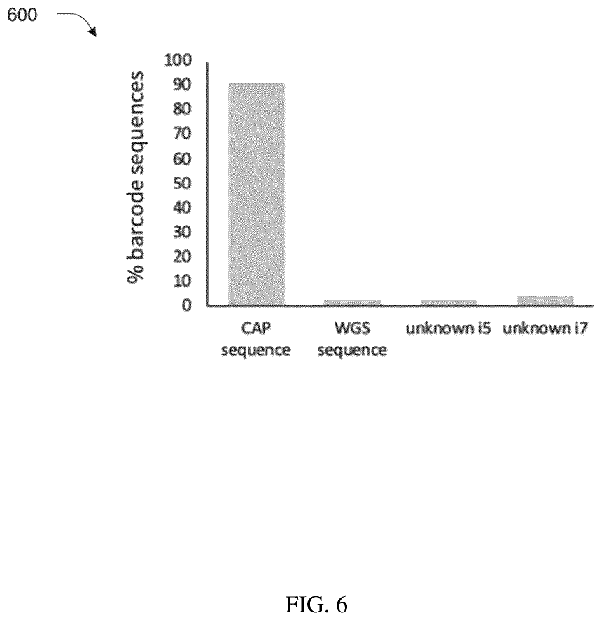

Bar graph 600 of FIG. 6 shows a distribution of reads for the first and the second sequence identifier tags (TAG1=“WGS” and TAG2=“CAP”) in the eight capture libraries after demultiplexing. As the results are for the capture libraries, in the case of failed conversion, only TAG1 tags (“WGS”) would be observed in the sequencing reads. In the case of successful conversion of all TAG1 tags (“WGS”) into TAG2 tags (“CAP”) during the post-capture amplification, only “CAP” sequences would be expected. The bar graph 600 also shows unknown i5 or i7 sequence due to errors in the barcode primers. The number of the unknown i7 (8 base pairs+5 base pairs long) is proportionally larger to the i5 (8 base pairs). Sequencing errors in i5 and i7 are as common as the occurrence of remaining WGS sequences in the run. The high percentage of “CAP” sequences indicates a high conversion rate.

### Example 2: Comparison of Different Capture Library Conversion Workflows

This example provides a comparison between two workflows.

The first workflow corresponds to conversion of capture library pool using a combination of non-proofreading polymerase (such as Klenow Fragment) and proofreading polymerase (HiFi Polymerase).

The second workflow corresponds to conversion of capture library pool using non-proofreading enzyme (such as Taq polymerase).

Barcode conversion rates and error rates of the first and the second workflows using the method 200 of the present disclosure were compared with a control capture experiment with standard amplification using HiFi Polymerase and P5/P7 sequencing primers without the sequence identifier tags.

The number of reads with either TAG1 (“W”) or TAG2 (“C”) barcodes was recorded for each capture library. In the case of failed conversion, only TAG1 tags (“W”) are expected. As the results are for capture libraries, in the case of successful conversion of all TAG1 tags into TAG2 tags, only TAG2 tags (“C”) are expected.

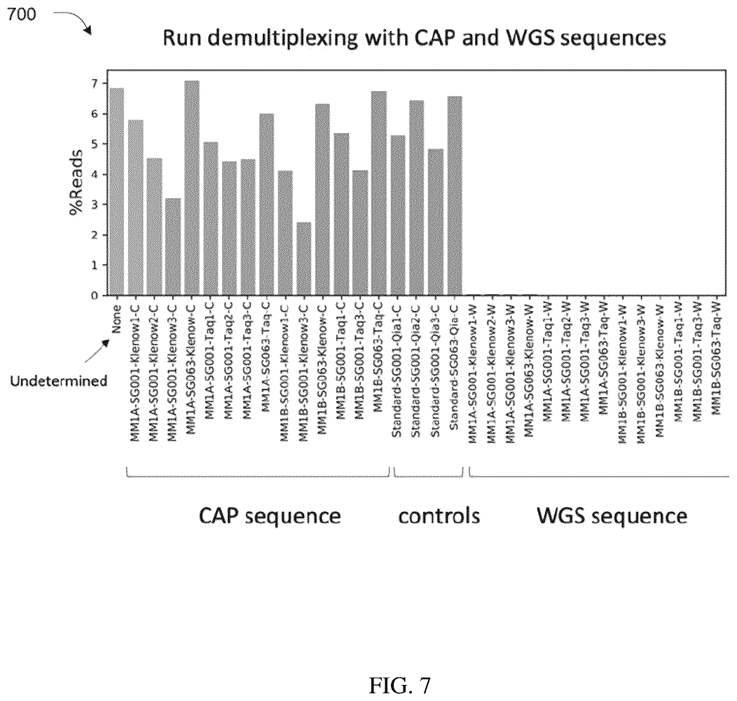

FIG. 7 shows the percentage of reads, out of the total number of reads in the sequencing runs, that are assigned to the different samples and that have either TAG1 (“W”) or TAG2 (“C”). Both the first and the second workflows show high conversion rates of the sequence identifier tags (TAG1, TAG2), as shown in bar graph 700 of FIG. 7. Additionally, two different combinations of the sequence identifier tag TAG1 and TAG2 barcoded primer versions are used in this experiment, named MMIA (TAG1a/TAG2a, as described in Example 1) and MMIB (TAG1b/TAG2b, as described in Example 1). The number of undetermined reads is in the expected range of the sequencer (5-10%, including 2% PhiX).

Error rates of the two workflows and control workflow were further compared by counting variants at low variant allele frequency (<10%) in captured libraries of reference samples (SG001, SG063). Captures were performed with a panel enriching for a target footprint of 156,274 base pairs.

For both primer versions (MMIA=TAG1a/TAG1b and MM1B=TAG2a/TAG2b), the second workflow (Taq Polymerase; “Taq”) shows the expected increase in low variant fractions compared to the control workflow, which is the expected result of amplification with a non-proofreading polymerase.

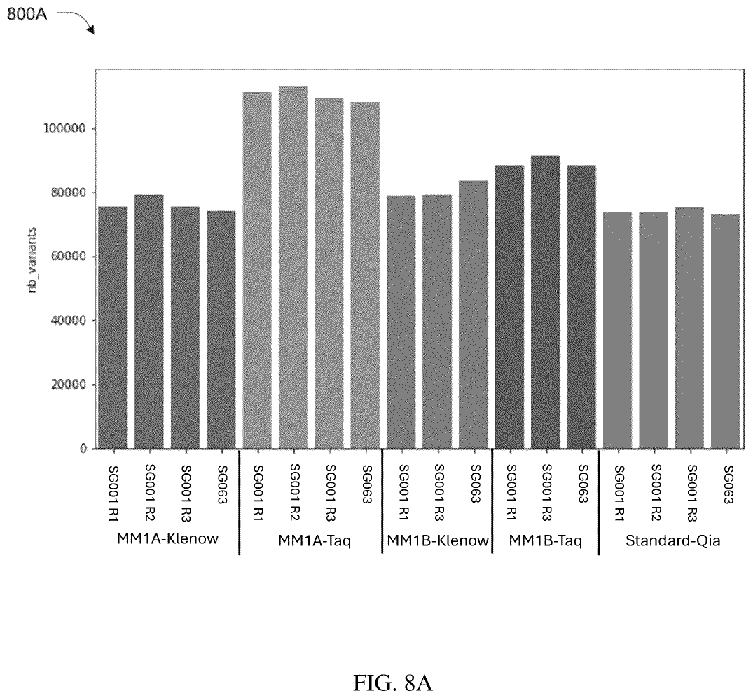

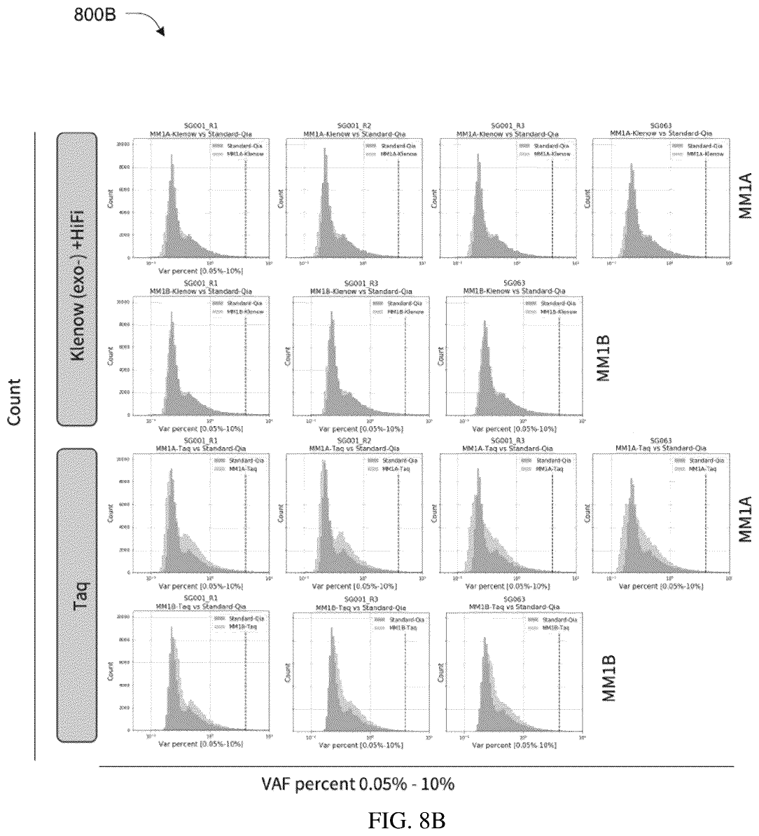

The first workflow (combination of enzymes; “Klenow”) shows a reduced error rate compared to the second workflow, which is comparable to the levels observed for the control workflow (“Standard-Qia”), as shown in bar graph 800A and histograms 800B in FIGS. 8A and 8B, respectively. Both figures show variants below 10% variant fraction for two references samples (SG001, SG063), with replicates for SG001, in all three workflows.

The method 200 described in the present disclosure may be used for implementation applications where WGS library pools are to be processed differently for different applications.

For example, the method 200 may be used to produce a combination of WGS and capture sequencing data for a given sample. In a medical context, the method 200 may allow WGS insights to be combined with detailed analysis of candidate (targeted) genes potentially linked to a given condition. In particular, establishing whether a given cancer is linked to homologous-recombination deficiency (HRD) helps predict the likely response to specific treatment options. The HRD status can be established by a combination of mutations in specific genes (especially BRCA 1/2) and genome-wide patterns indicative of frequent duplications or losses or large chromosomal segments.

### Example 3: Utilization of a Variety of Capture Probes

In an example, two sets of probes were designed to capture different sets of genes. Probes A represent a clinical exome solution and target 6380 genes. Probes B represent a custom panel and target 128 genes of special clinical interest or encompassing difficult markers, such as variants representing a combination of single-nucleotide variants in highly similar paralogs. The two sets of probes were mixed at four different relative concentrations.

In an embodiment, eight different samples were processed for each of the four probe mixes. For each sample, a WGS library was prepared with method 200. The DNA sample was fragmented, end-repaired and A-tailed before ligating adapters.

After post-ligation clean-up, indexing primers binding to the adapters were exploited to PCR amplify adapter-ligated DNA fragments and barcode each sample. After clean-up, the result was the WGS library.

Moreover, pools of eight barcoded WGS libraries were used for target enrichment, wherein each of the probe mixes were utilized once.

Denatured DNA was hybridized to the probes, and probe-target duplexes were then bound to streptavidin beads, which were pulled down and washed using standard protocols.

The captured DNA fragments were then PCR amplified and cleaned up, to produce the final capture library. The capture libraries were sequenced on an Illumina® Nextseq 2000 sequencer, as paired-end, 151 bp reads.

Next, each group of the eight samples captured with the same probe mix were treated as a single sample for the purpose of analyses. Reads were then cleaned and mapped to a reference genome. The median coverage was computed for regions corresponding to the clinical exome solution (i.e., Probes A), the part of the regions targeted by Probes B also covered by some Probes A (126 genes), and two genes targeted by Probes B, but not targeted by Probes A.

As a result, the coverage of regions targeted by Probes A remained approximately constant, while the coverage of regions targeted by Probes B increased proportionally to the ratio of Probes B to Probes A. The coverage of regions targeted by both Probes A and Probes B were higher than those targeted solely by Probes B.

Additionally, where Probes B (ratio of 0) is absent, no coverage was achieved for the latter, while the former had a coverage similar to the regions targeted solely by Probes A, as expected. The observed ratios will be correlated with the predicted ratios.

TABLE 3
Probe concentration ratios for genes covered in Probes B
(ratio 1) and those covered in Probes B and A (ratio 2).

| Expected | Expected | Observed | Observed |
|---|---|---|---|
| ratio 1 | ratio 2 | ratio 1 | ratio 2 |
| 0 | 1 | 0 | 0.90 |
| 1 | 1.3 | 1.23 | 1.69 |
| 2.75 | 3.1 | 2.88 | 3.36 |
| 6.7 | 7 | 4.61 | 5.83 |

Referring to Table 3, the observed ratios observed in the preliminary experiment are indicated for 84 genes covered in both Probes B and Probes A (Observed ratio 2) and 2 genes only covered in Probes B (Observed ratio 2).

Furthermore, a mix of Probes A and Probes B were utilized, wherein Probes B may have a per-probe concentration that is approximately 2.75 higher than those from Probes A. Considering that some genes are targeted by Probes B, but also some of Probes A, the predicted ratio for genes targeted by Probes B were approximately 3.1 higher than for genes targeted solely by Probes A.

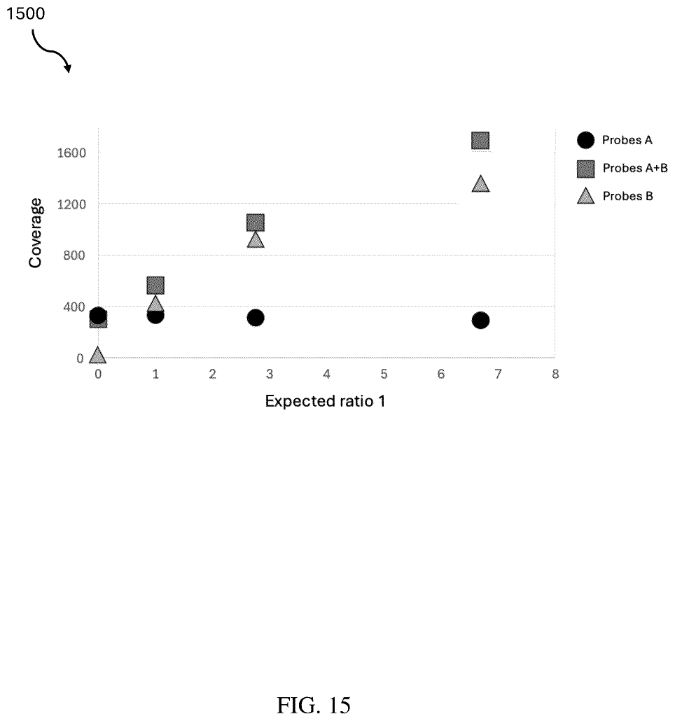

Referring to FIG. 15, a scatter plot 1500 of coverage obtained with different ratios of Probes B to Probes A (Ratio 1), for genes targeted solely by Probes A (circles), genes targeted solely by Probes B (triangles), and genes targeted by both Probes A and Probes B (squares) is depicted.

Separately, a total of 24 samples, corresponding to a diversity of reference samples, were selected. A WGS library was prepared using method 200. The DNA samples were fragmented, end-repaired and A-tailed prior to ligating adapters. After post-ligation clean-up, indexing primers binding to the adapters were employed to PCR amplify adapter-ligated DNA fragments and barcode each sample. After clean up, the result was the WGS library.

A total of 1,021,756,060 reads were obtained, of which 96.06% were mapped. Per sample, a median of 41,241,154 mapped reads were obtained (standard deviation=3,126,226). A good coverage uniformity was obtained, but with regions targeted by Probes B at higher coverage than those targeted by Probes A.

As a result, on average, 94% of the mapped reads mapped to the genes targeted by Probes A, in turn, leading to a median coverage per sample ranging from 190 to 273 for said regions. Despite a smaller number of reads, the coverage for regions targeted by Probes B ranged from 591 to 849, which reflects a higher relative concentration of Probes B in the probe mix.

On average, regions targeted by Probes B provided coverage that is more than three-fold higher than those targeted by Probes A (median=3.13, standard deviation=0.05). Despite the differences in the median coverage between regions targeted by Probes A and Probes B, the coverage was highly homogeneous within each of these categories (fraction of target regions within 20% and 500% of the median coverage; for Probes A, median among samples=0.989, standard deviation >0.001; for Probes B, median among samples=0.999, standard deviation >0.001).

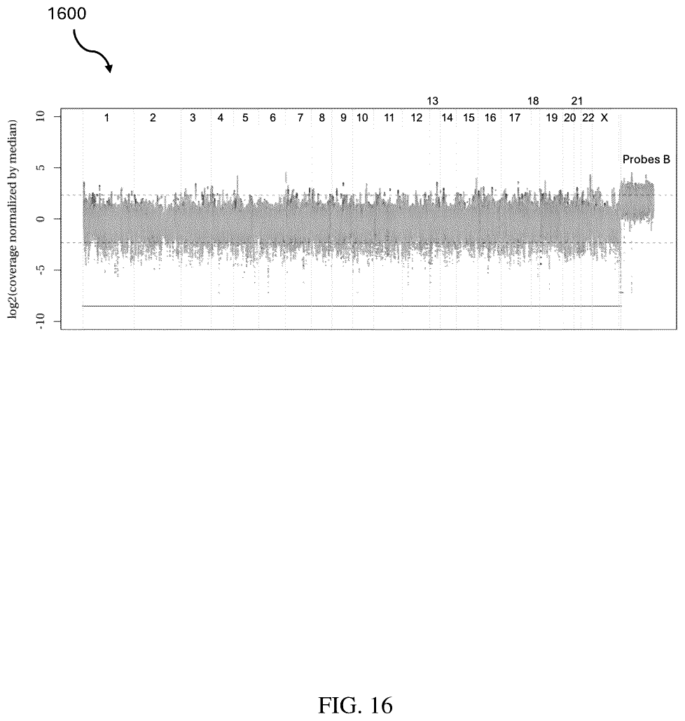

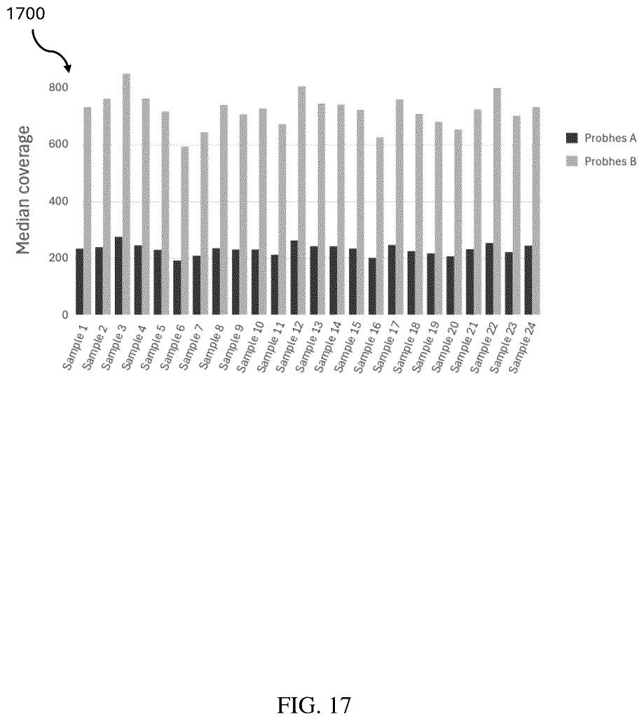

FIG. 16 depicts a graph 1600 showing coverage distribution among 50 kb windows, for one sample. In FIG. 16, the coverage of regions targeted by Probes A are plotted along the chromosomes, while those targeted by Probes B are grouped on the right.

FIG. 17 depicts a bar graph 1700 of a median coverage achieved for targets corresponding to two sets of probes A and B mixed before capture target-enrichment. The median coverage is shown for each set of probes corresponding to its respective sample.

The sequencing data was down-sampled to at least one of 9 million fragments and 20 million fragments. Upon down-sampling, the sequencing data was analyzed with bioinformatic pipelines pre-existing in SOPHIA DDM™. However, any suitable bioinformatic pipeline alternative can be utilized. Additionally, the analytical performance for SNVs and indels may have a sensitivity above 99%, and more specifically, above 99.99%, as achieved with a clinical exome sequencing panel, alone.

Further, star alleles were called using the pipelines available as part of SOPHIA DDM™ for Pharmacogenomics. Furthermore, the same pipeline was applied to 13 samples for which data was produced, but also previously with the use of clinical exome solution (SOPHIA DDM™ Clinical Exome Solution v3) and with the use of the SOPHIA DDM™ for Pharmacogenomics panel.

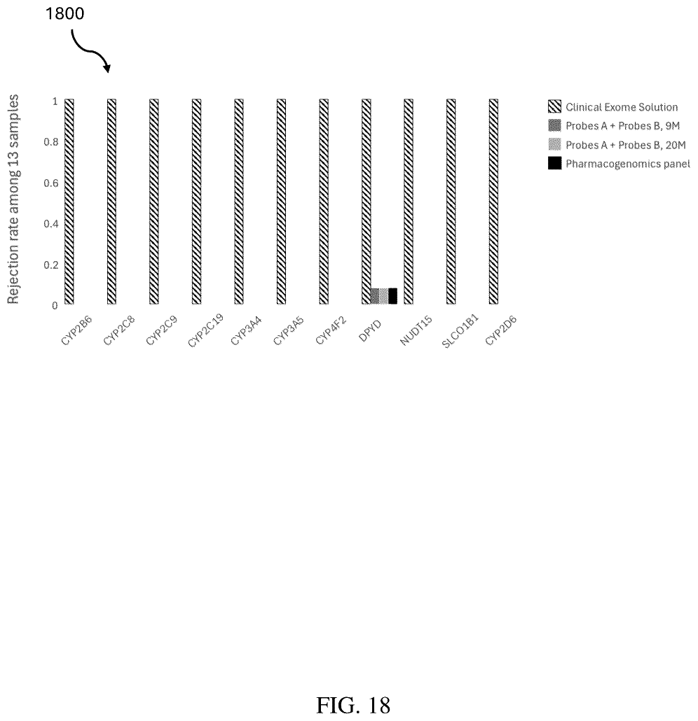

FIG. 18 displays a bar graph 1800 of a rejection rate for star allele calling. The rejection rate among 13 samples is plotted for each of 11 markers, each based on data obtained with a Clinical Exome Solution, with the invention (Probes A+Probes B) down-sampled to either 9 million fragments (9M) or 20 million fragments (20M) and with a pharmacogenomics panel.

Additionally, all of the samples were rejected based on data from a clinical exome solution due to insufficient coverage and high heterogeneity, and only one marker from one sample was rejected with the pharmacogenomics panel data, as show in bar graph 1800 of FIG. 18.

As a result, the data produced, with a combination of Probes A and Probes B, can achieve the same rejection rate as the data from the pharmacogenomics panel. The low rejection rates demonstrate the usefulness of the systems and methods for resolving star alleles while simultaneously benefitting from the genotyping of numerous genes.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A method for tagging one or more sequencing libraries derived from one or more DNA fragments of a DNA sample, comprising:
adding an adapter to a plurality of DNA fragments of the DNA sample to obtain an initial library pool; and
incorporating a sequence identifier tag to the at least one adapter of one or more subsets of sequencing libraries from the initial library pool.

2. The method of claim 1, further comprising:
denaturing the DNA fragments;
hybridizing the denatured DNA fragments with primers;
amplifying the DNA fragments to obtain the initial library pool;
amplifying the one or more subsets of sequencing libraries;
pooling and sequencing the one or more subsets of sequencing libraries;
demultiplexing each of the one or more subsets of sequencing libraries based on a corresponding sequence identifier tag;
producing a capture library pool of at least one targeted DNA sequences from the one or more subsets of sequencing libraries using targeted sequencing techniques; and/or
amplifying the capture library pool.

3. The method of claim 1, wherein the sequence identifier tag is incorporated using a polymerase and primers, and wherein the sequence identifier tag is placed between an index sequence and an at least one oligonucleotide sequence of the at least one adapter.

4. The method of claim 3, wherein the primers are mismatching primers.

5. The method of claim 3, wherein the at least one oligonucleotide sequence is a P7 primer and the index sequence is an i7 index.

6. The method of claim 1, wherein the sequence identifier tag has between 5 to 12 base pairs.

7. The method of claim 1, wherein the at least one adapter added to the DNA fragment comprises an initial sequence identifier tag, and wherein for incorporating the sequence identifier tag, the method comprises modifying the initial sequence identifier tag to the sequence identifier tag.

8. The method of claim 7, wherein the initial sequence identifier tag matches the sequence identifier tag on the 5′ end, and wherein the initial sequence identifier tag differs from the sequence identifier tag by at least two bases at the 3′ end.

9. The method of claim 2, wherein the amplifying the capture library pool uses at least one of: (i) a first polymerase lacking 3′ to 5′ exonuclease proof-reading activity, (ii) and/or a second polymerase that is a proof-reading polymerase.

10. The method of claim 2, wherein for amplifying a template DNA fragment from the capture library pool, the method comprises:
maintaining a temperature of the template DNA fragment below a first temperature threshold for a first time period to activate the first polymerase; and
maintaining the temperature of the template DNA fragment above a second temperature threshold for a second time period to activate the second polymerase.

11. The method of claim 1, wherein the DNA sample comprises genomic DNA, comprises cDNA synthesized from RNA, or cell-free DNA (cfDNA) isolated from a bodily fluid, such as blood, urine, or cerebrospinal fluid.

12. A method for analyzing various regions of a genome at different resolutions, the method comprising:
producing, a WGS library,
wherein the WGS library is created from at least one of DNA, cfDNA, RNA, and TNA;
enriching, the WGS library, for each of one or more regions of interest to produce a capture sequencing library;
wherein a first grouping of genomic regions are represented at a higher coverage than a second grouping of genomic regions;
sequencing, with next generation sequencing, the pooled sequencing library, creating genetic data; and
analyzing, the genetic data, to identify genetic markers.

13. The method of claim 12, wherein:
the capture sequencing library and the WGS library are pooled prior to sequence to produce a pooled sequencing library;
sequence identifiers are integrated in the adapters and wherein the sequence identifiers differ between the capture and WGS libraries;
the sequence identifier was modified in the capture library through post-capture amplification with a mismatching primer;
the sequence identifier was modified in the WGS library through PCR amplification with a mismatching primer after aliquoting part of the WGS library to produce the capture library; and/or
the probes used to produce the capture library include a whole exome sequencing panel, a clinical exome sequencing panel, a comprehensive genomic profiling panel, or a small, targeted panel including genes linked to a condition of interest.

14. A method for demultiplexing one or more sequencing libraries, the method comprising:
ligating at least one adapter to a DNA fragment, wherein the DNA fragment is associated with a DNA, cfDNA, RNA, or TNA sample, to obtain a WGS library;
incorporating a sequence identifier tag to the at least one adapter to one or more subsets of sequencing libraries, wherein the one or more subsets of sequencing libraries is derived from the WGS library;
pooling the one or more subsets of sequencing libraries;
sequencing the one or more subsets of sequencing libraries; and
demultiplexing each of the one or more subsets of sequencing libraries based on the corresponding sequence identifier tag.

15. The method of claim 14, further comprising:
denaturing the DNA fragments;
hybridizing the denatured DNA fragments with primers;
amplifying the DNA fragments to obtain the first sequencing library;
amplifying the one or more subsets of sequencing libraries; and/or
producing a capture library pool of at least one targeted DNA sequences from the one or more subsets of sequencing libraries using targeted sequencing techniques; and
amplifying the capture library pool.

16. The method of claim 14, wherein:
the sequence identifier tag is incorporated using a polymerase and primers;
the primers are mismatching primers;
the sequence identifier tag is incorporated between an index sequence and an at least one oligonucleotide sequence of the at least one adapter;
the at least one oligonucleotide sequence is a P7 primer and the index sequence is an i7 index; and/or
the sequence identifier tag has between 5 to 12 base pairs.

17. The method of claim 14, wherein the at least one adapter added to the DNA fragment comprises an initial sequence identifier tag, and wherein for incorporating the sequence identifier tag, the method comprises modifying the initial sequence identifier tag to the sequence identifier tag.

18. The method of claim 17, wherein the initial sequence identifier tag matches the sequence identifier tag on the 5′ end, and wherein the initial sequence identifier tag differs from the sequence identifier tag by at least two bases at the 3′ end.

19. The method of claim 15, wherein the amplifying of the capture library pool uses at least one of: (i) a first polymerase lacking 3′ to 5′ exonuclease proof-reading activity, (ii) and/or a second polymerase that is a proofreading polymerase,
wherein for amplifying a template DNA fragment from the capture library pool, the method comprises:
maintaining a temperature of the template DNA fragment below a first temperature threshold for a first time period to activate the first polymerase; and
maintaining the temperature of the template DNA fragment above a second temperature threshold for a second time period to activate the second polymerase.

20. The method of claim 14, wherein:
the capture sequencing library is produced with probes comprising a whole exome sequencing panel, a clinical exome sequencing panel, or a comprehensive genomic profiling panel;
the capture sequencing library is produced with probes comprising a small panel targeting genes linked to a given condition;
the probes used to produce the capture library comprise of mixture of at least two sets of probes targeting different genomic regions, wherein the two sets of probes are present at different relative concentrations, so that some regions are more representing in the sequencing reads and/or wherein the genomic regions targeted by the two sets of probes overlap.
